(19) United States
(12) Patent Application Publication
(10) Pub. No.: US 2023/0004817 A1
(43) Pub. Date: Jan. 5, 2023

(54) APPARATUS AND METHOD FOR DYNAMIC VISUALIZING AND ANALYZING MICROBIOME IN ANIMALS

(71) Applicant: DSM IP Assets B.V., HEERLEN (NL)

(72) Inventors: Ghislain SCHYNS, Columbia, MD (US); Britta BLOKKER, Columbia, MD (US); Joshua CLAYPOOL, Columbia, MD (US)

(21) Appl. No.: 17/779,959

(22) PCT Filed: Nov. 23, 2020
(86) PCT No.: PCT/US2020/061787

§ 371 Date: May 25, 2022

Related U.S. Application Data
(60) Provisional application No. 62/941,557, filed on Nov. 27, 2019.

(51) Int. Cl. G06N 3/12; G06T 11/20; G16B 45/00

(52) U.S. Cl. CPC G06N 3/126; G06T 11/206; G16B 45/00

(57) ABSTRACT

A method for visualizing microbiome data is described. Respective microbes and/or genes in microbiome data stored In in a database are identified. A network comprising nodes interconnected by edges is generated in a memory of a computer, each node representing one or more identified microbes or one or more microbial metabolites, and each edge of the network representing an association between a respective pair of the one or more identified microbes or a reaction mediated between two metabolites by an enzyme encoded in the one or more identified genes, with at least some nodes and edges of the network being each associated with a condition attribute identifying a groups and/or a timestamp associated with a sample in the database. The displayed network is dynamically updated in accordance with a filtering of the microbiome data based on the condition attributed and/or the timestamp attributed. Corresponding systems and computer-readable storages are also described.

### CROSS-REFERENCE TO RELATED APPLICATIONS

This application claims the benefit of the filing date of U.S. Provisional Patent Application No. 62/941,557, filed Nov. 27, 2019, the disclosure of which is hereby incorporated by reference in its entirety.

### FIELD OF THE TECHNOLOGY

The technology presented herein relates to systems and methods related to visualizing digitized microbiome data, which can include visualizing digitized multi-omic host animal data.

### BACKGROUND

The genetic material of an animal microbiomes holds a large amount of information. Metagenomics is the study of genetic material from environmental samples, such as animal microbiomes. The obtaining of data from genetic material is typically performed by a “pipeline”. Current pipelines deliver data with no regard to where and how the sample was derived. Such data requires significant amount of processing before it can be interpreted by a scientist.

Information from genetic material of animals are used for applications such as diagnostics and drug discovery. The more information that can be obtained from such material regarding the effects of treatments and the like, the better it is for such uses.

Thus, there is a need for systems and methods capable of visualizing digitized microbiome data from animals in a format that allows a scientist to interpret the data and reach actionable conclusions, including, but not limited to the identification of biomarkers and therapeutic targets.

### SUMMARY OF EMBODIMENTS

Some embodiments provide interactive visualizations that have been re-associated with the data from which the samples were collected to produce easily interpretable and interactive results.

A method for visualizing microbiome data is described. Respective microbes and/or genes in microbiome data stored in a database are identified. A network comprising nodes interconnected by edges is generated in a memory of a computer, each node representing one or more identified microbes or one or more microbial metabolites, and each edge of the network representing an association between a respective pair of the one or more identified microbes or a reaction mediated between two metabolites by an enzyme encoded in the one or more identified genes, with at least some nodes and edges of the network being each associated with a condition attribute identifying a groups and/or a timestamp associated with a sample in the database. The displayed network is dynamically updated in accordance with a filtering of the microbiome data based on the condition attributed and/or the timestamp attributed. Corresponding systems and computer-readable storages are also described.

### BRIEF DESCRIPTION OF THE DRAWINGS

FIG. 1 illustrates an example display of a microbiome visualization system, according to some embodiments.

FIG. 2 illustrates an example computer of a microbiome visualization system, according to some embodiments.

FIG. 3 is a flowchart of a process for obtaining microbiome data for visualizing and analyzing using the system of FIG. 2, according to some embodiments.

FIG. 4 is a visualization of taxonomic data from microbiome data according to a conventional system. The conventional visualization is a non-metric multi-dimensional scaling (NMDS) plot of all samples within a control and treatment samples. Each point represents the entire collection of microbes within the sample and the different shape represent the control (▪) and the treatment (▴).

FIG. 5 is a flowchart of a process for taxonomic visualizing and analyzing microbiome data using the system of FIG. 2, according to some embodiments.

FIG. 6 (FIGS. 6A, 6B, 6C, 6D, and 6E) shows respective display screens during a visualization and analysis using the process of FIG. 5, according to some embodiments. FIG. 6A shows the scaffold of all organisms. No edges/connections have been included and therefore this network currently has no structure. FIG. 6B shows, for the control case, organisms that were found to co-vary (Spearman r≥0.6) with an edge drawn between them. These edges created ball-like structures within the network. FIG. 6C shows, for the treatment case, organisms that were found to co-vary (Spearman r≥0.6) have an edge drawn between them. These edges created ball-like structures within the network. The network had a clearly different structure after a treatment was applied. FIG. 6D shows the distribution of connectivity of organisms within the network that experienced the largest shifts (e.g. ZiPi) distance. Increasing P1 shows an increase in connectivity to multiple groups in the network and an increasing Z1 shows an increasing connectivity to an individual group. FIG. 6E shows when network structure was evaluated for ‘small-worldedness’ by the factor S, where an S>>1 indicates a small-world effect. The strength of the small-world effect increased with treatment. This indicates that more organisms/nodes were tightly connected after the treatment than before.

FIG. 7 is a visualization of functional data from microbiome data according to a conventional system. The illustrated heatmap represents the relative abundance of KEGG modules (defined by KEGG database) for all samples.

FIG. 8 is a flowchart of a process for functional visualizing and analyzing microbiome data using the system of FIG. 2, according to some embodiments.

FIG. 9 (FIGS. 9A, 9B, and 9C) shows respective display screens during a visualization and analysis using the process of FIG. 8, according to some embodiments.

FIG. 9A shows a network generated using information from a public database (KEGG) where dots/nodes are metabolites, and connections/edges are enzymes or known reactions. The width of each reaction is normalized to a control condition. In FIG. 9B edges show a change in abundance relative to a control condition, where reactions in the upper left bordered area see a decrease, whereas reactions in the middle lower bordered area see a large increase. In FIG. 9C, information relating to the statistical significance of the change in abundance of the reactions between metabolites was overlaid onto the network. This shows the statistical significance of the change in the reaction relative to the control as a function of color (black->white or darker->lighter).

FIG. 10 is a flowchart of a process for integrated functional and taxonomic visualizing and analyzing microbiome data using the system of FIG. 2, according to some embodiments.

FIG. 11 (FIGS. 11A, 11B, 11C, 11D and 11E) shows respective display screens during a visualization and analysis using the process of FIG. 10, according to some embodiments. In FIG. 11A, the relative abundance of two organisms are shown in a control group (0) and in treatment groups (Low, High) that received treatment with different doses of an enzyme. There is a large change in the relative abundance of the two organisms due to the dose of the applied treatment. FIG. 11B shows the initial functional network considering these two organisms as operating together as a supra-organism. The detected genes/enzymes are represented by grey lines connecting the potential metabolites represented as black dots. Three metabolites in bordered area (highlighted) were selected for further focus. FIG. 11C shows focusing on the three metabolites in the bordered area in FIG. 11B, it appeared the reactions, when treated, between two of them are getting thicker relative to the control. The individual effect of each organism was not known in this situation. FIG. 11D shows, using a network created for each organism, the genes were mapped accordingly. Organism 1, represented on the left, and organism 2, represented on right. FIG. 11E shows, focusing on the three metabolites from the bordered area in FIG. 11B, under control conditions (left panel), organism 1 to have both genes catalyzing the reactions between all three metabolites, where organism 2 only has one gene. Under high dose of enzymes (right panel), organism 2 genes are seen to increase but there is a general decrease in organism 1.

FIG. 12 is a flowchart of a process for identifying genes and/or metabolites for diagnostics relative to control condition using the system of FIG. 2, according to some embodiments.

FIG. 13 (FIGS. 13A, 13B, 13C, 13D and 13E) shows respective display screens during a visualization and analysis using the process of FIG. 12, according to some embodiments. FIG. 13A shows a base metabolic network for the desired state (no diarrhea). All detected edges were normalized to this state. FIG. 13B shows, upon shifting to a worsening condition (severe diarrhea), a visualization of the decrease in pathways in upper left bordered area, and increase in other reactions in lower right bordered area. FIG. 13C=shows, available metabolite data embedded to increase the information derived from the dynamic metabolic network (not all metabolites were measured). Control state of the network (no diarrhea) is shown. The area where a decrease in some reactions was seen is highlighted with the bordered area. FIGS. 13D and 13E. Zoomed in on the boxed area from FIG. 13C, the control (no diarrhea) case is shown with measured metabolites highlighted (FIG. 13D). By visualizing the dynamic network under severe diarrhea conditions (FIG. 13E), the change in abundance of the metabolite upstream of the missing enzymes is seen. The decrease in the metabolite downstream, and right at the inflection point in gene abundance, suggests both a direction of metabolite flow and what genes are important for the accumulation and dissipation of the increasing metabolite.

FIG. 14 is a flowchart of a process for using the system of FIG. 2 to develop standards to monitor fluctuations in microbiome, according to some embodiments.

FIG. 15 (FIGS. 15A, 15B, 15C and 15D) shows respective display screens during a visualization and analysis using the process of FIG. 14, according to some embodiments. FIG. 15A shows a metabolic network established in a control state. A metabolite of interest is highlighted with an increased size of the node/metabolite. FIG. 15B shows that, compared to control conditions, there was an edge increase due to a specific treatment that had a positive effect on the host. An adjacent highlighted node is included in the upper right corner for frame of reference. FIG. 15C shows a ranking of standard deviation of all edges/genes/reactions. FIG. 15D shows using an internal edge with low standard deviation as a comparison, relative abundance of candidate marker is shown. Treatments B and D increased expression of the candidate marker.

FIG. 16 shows proline metabolic network of a supra-organism comprising the host and its microbiome. Red and blue boxes indicate host and microbiota genes/enzymes, respectively. The white boxes indicate genes/enzymes not being detected in the samples.

FIG. 17 (FIGS. 17A and 17B) shows that dynamic network can embed host responses and/or direct connections to metabolites to get a view of the strength and how the microbiome is connecting to the host. In FIG. 17A, the dynamic network embeds host responses and direct connections to metabolites to get a view of the strength and how the microbiome is connecting to the host. The host response to putrescine is represented by the largest red node and the molecule putrescine is represented by the large connected black node. As shown in FIG. 17B, the dynamic network can allow for a multi-dimensional view into the microbiome-host connection. The width of the lines between nodes is the gene abundance relative to the control, and the color gradient represents the q-value (lower is more significant). This shows in the treated case that the microbiome had significant connections to putrescine and this is reflected in the connection to the host.

### DETAILED DESCRIPTION

In one aspect, disclosed herein are systems and methods using a database capable to efficiently process vast amounts of data (e.g. nucleic acid data) generated from animal microbiome samples into visual displays that allow a user (e.g. scientist etc.) to rapidly and reliably detect changes in the taxonomic structure and/or functional organization of the microbiome under different conditions, identify genes and metabolites as candidate markers for conditions, such as emergence of disease state or response to therapy, identify therapeutic targets, such as genes and metabolites as targets for supplementation, and identify internal standards for diagnostic markers. The systems and methods described herein can integrate into the visual display additional data obtained from multi-omic analysis of the microbiome, such as genomic sequence, gene expression, and metabolite concentrations, as well as information related to the host organism, such as gene expression data from the host organism.

Some exemplary embodiments of this disclosure include a display and a computer system configured to facilitate the interactive viewing of microbiome data of animals. As used herein, the term “animal” includes all farm animals. The examples of the animals include non-ruminants and ruminants. Ruminants include but are not limited to sheep, goat and cattle; and non-ruminants include but are not limited to horse; rabbit; pig including but not limited to infant pig, piglet, growing-fattening pig, sow and boar; and poultry such as turkey, duck and chicken (including but not limited to broiler chicken, egg-laying chicken) etc. The microbiome data contains information on a large number of different microbes, metabolites etc., which all may be affecting the condition of the host animal. A clear understanding of the entire microbiome, or at least a substantial part of the microbiome, can be very valuable for purposes such as diagnostics, drug discovery, identifying or establishing standards, etc.

In contrast to many conventional systems and techniques for visualizing genetic information, embodiments of the present invention enable the viewing of the entire microbiome of the animal. Embodiments use dynamic networks to efficiently represent the microbiome. In the dynamic network, microbes (also referred to interchangeably in this disclosure as “organisms”) and/or metabolites are represented as nodes. The edges of the dynamic network may be based on one or more of a correlation between microbes, an enzymatic reaction that transforms one metabolite to another, and/or other association between the aspects represented in the nodes.

Some embodiments provide systems and methods to extract mode of action related to taxonomic and functional information from animal microbiome samples derived from in-vivo, in-vitro, and ex-vivo screening systems where at least two conditions (e.g., control condition, treatments) can be compared. In some embodiments, the input takes nucleic acid compositions, for example, from a massively parallel sequencer and runs the output sequence files through a series of computational algorithms to establish taxonomic and functional classification of the input sequence files. In some embodiments, the nucleic acid compositions are genomic DNA compositions. In some embodiments, the nucleic acid compositions are cDNA compositions. In some embodiments, the nucleic acid compositions are RNA compositions. In some embodiments, the input takes protein compositions, for example, from a proteomic analysis and runs the output sequence files through a series of computational algorithms to establish taxonomic and functional classification of the input protein sequence files. The taxonomic output is associated with experimental metadata and converted into an interactive visualization. The functional output is referenced against a metabolic network. Relevant genomic fluctuations, statistics, enzyme information, compounds, treatments can be embedded into this dynamic network. The network can then be interactively visualized to show differences among the respective conditions. These techniques can be applied to both shotgun metagenomes (e.g., fragmented DNA sequenced using 2D reads) or shallow shotgun metagenomes (e.g., fragmented DNA sequenced using 1D reads).

As noted above, embodiments process input data (such as DNA, RNA or cDNA sequencing reads from a high-throughput sequencer or high-throughput protein data), and generate visualizations of taxonomic and functional changes between at least two samples derived from a similar environment but from differing conditions.

Some embodiments, may quantify genes to reactions identified between two compounds irrespective of a classified pathway. A re-association of sample metadata back into the process is made in order to allow for more useful visualization. This pipeline allows the flexibility to embed statistics and other metadata into the visualizations to facilitate simultaneous analysis across multiple dimensions.

In some embodiments, a gene catalog allows sufficient depth to properly observe function and make the statistical claims embedded in the functional visualizations. The use of a marker-gene based taxonomic prediction in some embodiments improves taxonomic information over kmer-based assignment of reads for animal microbiome studies.

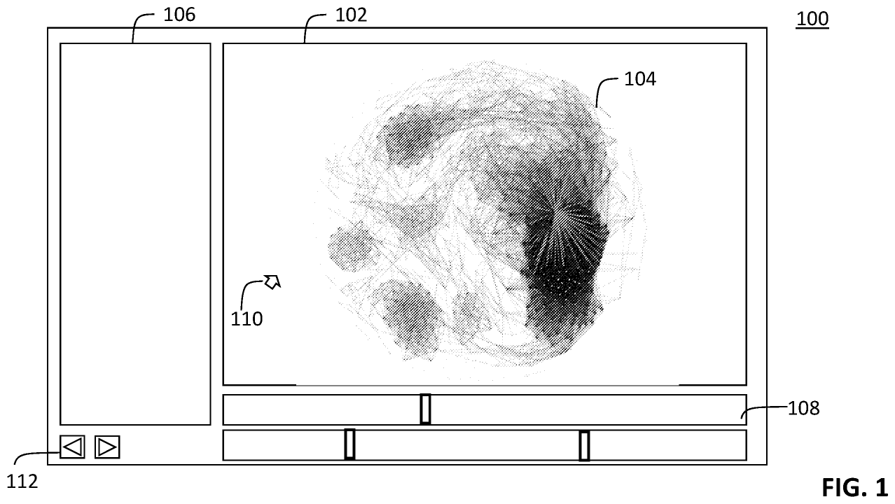

FIG. 1 illustrates a display 100 of a microbiome visualization system, according to some embodiments.

The display 100 includes a first display area 102 in which a dynamic network 104 corresponding to a microbiome, or apart thereof, is displayed based on the microbiome data corresponding to samples of genetic data collected from animals. The dynamic network 104 may be viewable in whole, so that the interactions of all the microbes, metabolites and/or enzymes that are present in the microbiome of the group of animals being investigated on a single screen, or may be viewable in part displaying only a part of the network containing certain microbes, metabolites, enzymes and/or their interactions of interest.

A second display area 106 may be used to display various information regarding the dynamic network and/or configuration of the visualization system. The second display area, for example, may be used to display calculated information such as various network statistics. Example network statistics that can be determined by the system by processing the data according to the dynamic network may include statistics such as eigenvector centrality (e.g., identifies an organism highly connected to its own sub-community) and betweenness centrality (e.g., identifies organisms highly connected to different sub-communities) with which a user can, among other things, identify keystone organisms. Keystone organisms are characterized by the connections they share to other organisms. There exist four general classifications of keystone organisms: network hubs, module hubs, connectors, and peripherals. These organisms are classically evaluated by a “Zi” parameter, a measure of connectedness to its own sub-community, and a “Pi” parameter, a probability that it is more highly connected to other sub-communities than to its own. Typical cut-offs for establishing a ‘hub’ is that is has a “Zi” value greater than 2.5. A connector has a “Pi” value greater than 0.62. Generalists have both a “Zi” greater than 2.5 and a “Pi” greater than 0.62 whereas peripheral organisms meet none of these requirements. For details on calculation of these parameters, see: https://www.ncbi.nlm.nih.gov/pmc/articles/PMC2148393/ (originally Z=Zi and c=Pi),

The display 100 may include one or more controls 108 to control the selection of data displayed in the dynamic network. In some embodiments, the microbiome data may be selected according to the group (e.g. control group, treatment group 1, treatment group 2, etc.) and/or sample timestamp (e.g., sample time 1, sample time 2, sample time 3, etc., acquired in time sequence). Controls 108 may provide a slider or the like to select from the available samples. In some embodiments, a separate slider 108 may provide for each sample selection dimension, e.g., one slider enables the user to select a group and a second slider allows the user to select the timestamp of the sample for which the dynamic network is to be displayed.

Additional controls such as a cursor 110 may be provided in some embodiments. The user may use the cursor to select one or more nodes, one or more edges, and/or an area of the dynamic network to be further investigated and/or expanded in the viewable area.

Still further, some embodiments may provide for controls 112 to initiate automatic playback, forwarding, rewinding etc., enabling the user to view the changes of the dynamic network as the respective dynamic networks corresponding to each available sample instance is brought into view in a continuous sequence.

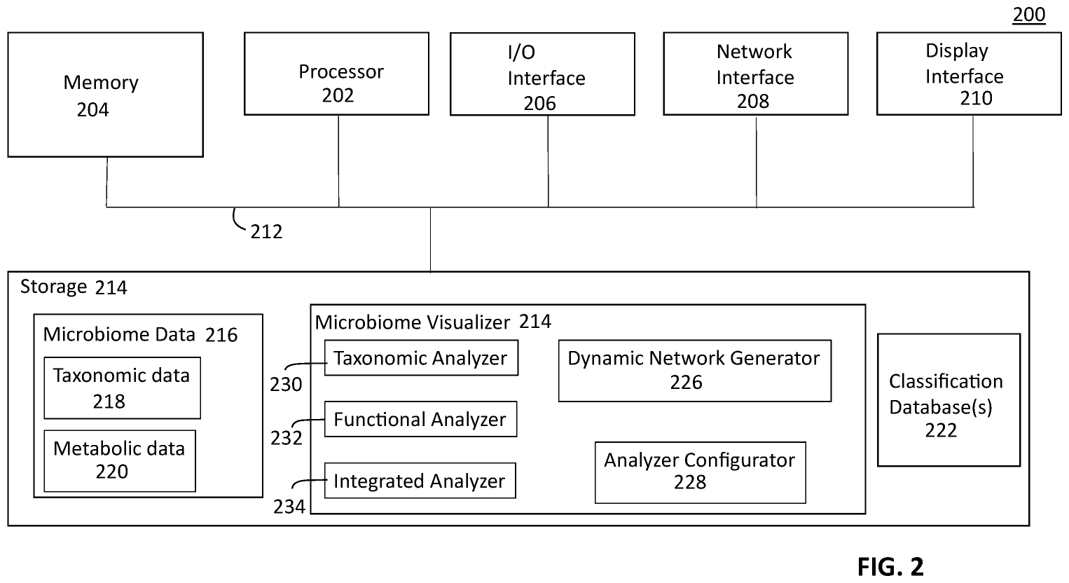

FIG. 2 illustrates an example computer 200 of a microbiome visualization system, according to some embodiments.

The computer 200 includes one or more processor 202, one or more memory 204, input/output interface(s) 206, network interface(s) 208, and display interface 210. A communication bus or other communication infrastructure 212 interconnects the components of the computer 200.

The processor 202 executes program instructions of a microbiome visualizer 224 from a memory 204 in order to access microbiome data 216 and, in some embodiments, classification databases 222 stored in storage 214 or at a network-connected location. As will be understood by persons skilled in the art, the data (e.g. microbiome data 216, classification databases 222, configuration settings 228, etc.) and program instructions (e.g. program code for microbiome visualizer 224) may be stored in a non-volatile storage 214 before being loaded into a volatile memory 204 by the processor 202 the time of running microbiome visualizer 224.

The display interface 210 may connect to a display such as the display 100 described above, enabling the dynamic network and other data generated by the processor to be displayed on the display and/for the user to interact with the displayed network and/or other information. The i/o interface 206 may connect to keyboard, mouse, touchscreen etc. to receive interactive input from a user.

The computer system 200 provides automated capabilities and interactive capabilities for a user, such as, for example, a scientist, a doctor, or an analyst, to visualize the microbiome of one or more host animals, in its entirety or in part in a manner in which interactions and/or effects between the different components of the microbiome can be visually observed. Automated capabilities may be provided by calculating selected network statistics (e.g. eigenvector centrality, betweenness centrality, modularity, sub-community detection analysis (as characterized by different sub-community detection algorithms, meta-analysis parameters extended from databases (such as number of previously observed occasions outside of the current study: e.g. Lactobacillus; mean relative abundance: 2.5%), p-values for treatments, average relative abundances, etc.) for the dynamic network of the microbiome, in order to either identify or highlight for the user aspects of interest in the microbiome. The interactive capabilities provide the user with the capability to control the displayed network to rapidly view the network or portions thereof in order to observe areas/aspects of interest of the microbiome. The computer system enables the visual comparison of the microbiome, more accurately, the visual comparison of data representing the microbiome, to view changes effected by treatments.

The storage 214 may be centralized or distributed and may include microbiome data 216, classification databases 222, and the microbiome visualizer 214.

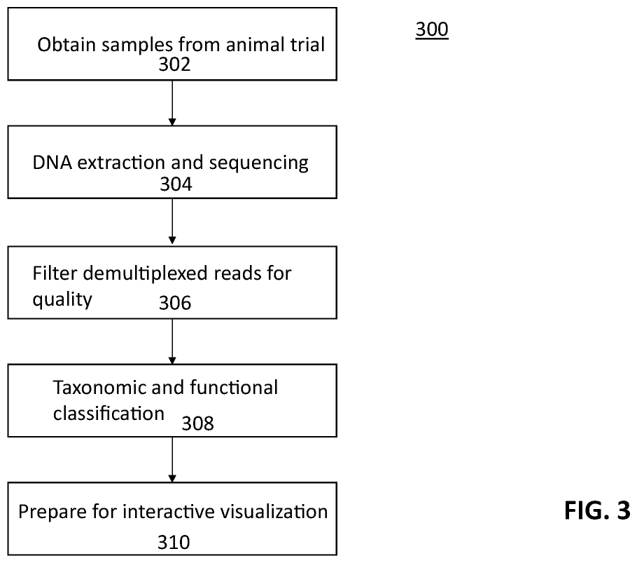

The microbiome data 216 may include both taxonomic data 218 and metabolic data 220. The microbiome data 216 may be obtained from a process pipeline such as that shown in FIG. 3. Quality control of sequencing is done on raw, demultiplexed reads to ensure proper removal of adapters and ensure appropriate length of sequences is maintained for downstream processing. In some embodiments, sequences are first examined and then trimmed using a sequence filtering software. The filtered reads can be further aligned against host DNA if no pre-filtered database exists downstream. This filtering may be done using a Burrow-Wheeler Alignment tool or the like, and by removing any read or read-pair that has mapping to the host genome.

The classification databases 222 may include one or more publicly available databases or custom databases. Some of the databases may be custom built around microbiome environments of interest and hand-curated to produce optimal results around understanding which genes are present. In some embodiments, function is classified using an alignment algorithm for the forward and reverse (if present) reads onto one or more classification databases. In some embodiments, a Burrows-Wheeler Alignment tool may be used.

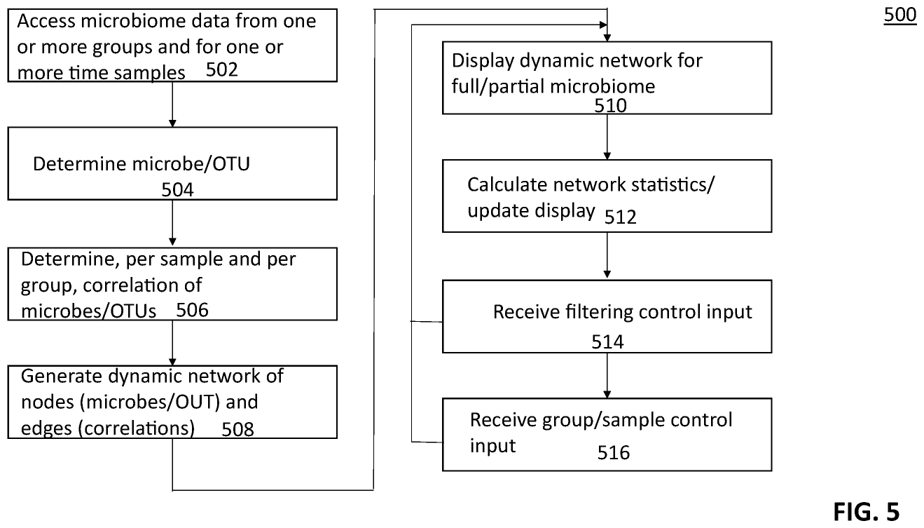

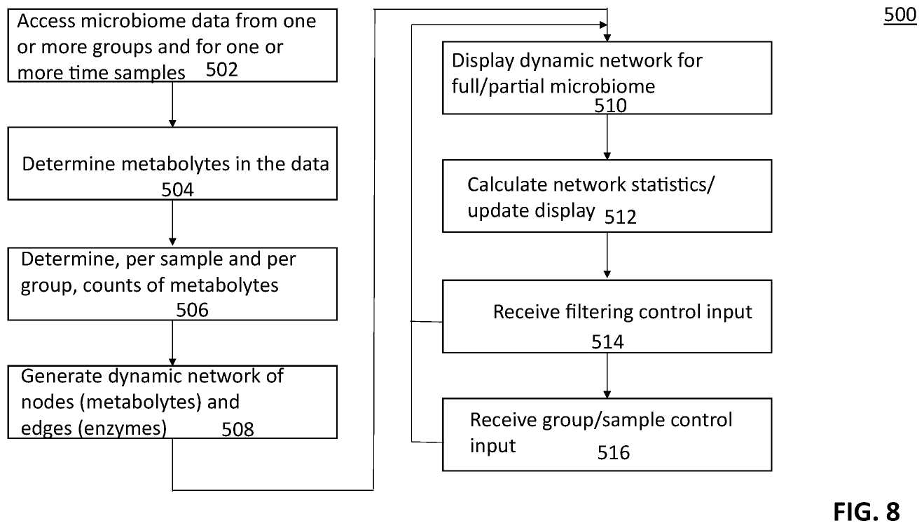

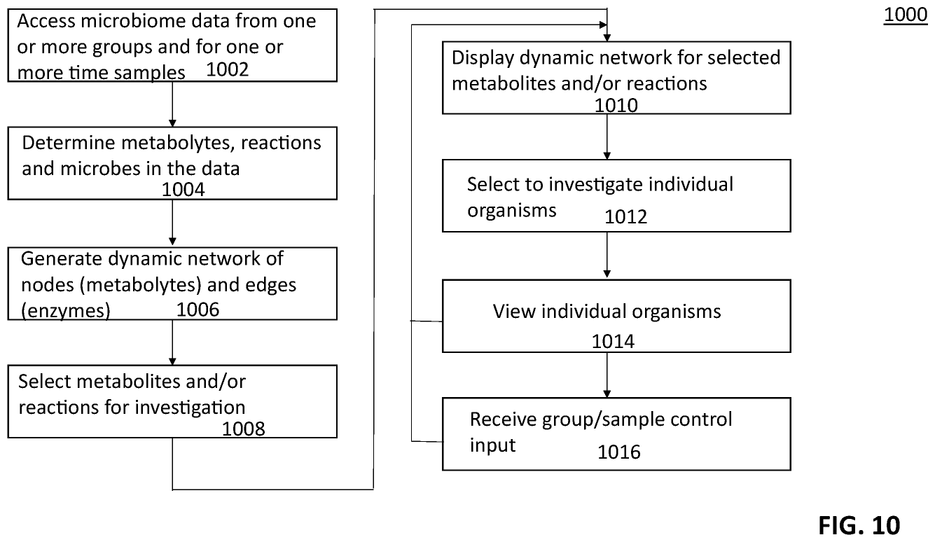

The microbiome visualizer 214 includes a dynamic network generator 226, and a configuration module 228. It also includes a taxonomic analyzer 230, a functional analyzer 232 and an integrated analyzer 234. The dynamic network generator 226 may operate to generate and maintain a dynamic network of nodes and edges created from microbiome information as described in this application. The configuration module 228 provides for user configuration of configuration parameters such as threshold values for automatically detecting potentially interesting relationships between the nodes and/or edges of the dynamic network (and thereby, the microbiome components represented therein). Configuration parameters may also include databases and the like to be used for certain analysis. The microbiome visualizer 214 includes program instructions and configurations for performing the processes and generating the display screens described in relation to FIGS. 5-6, and 8-17. Certain modules, such as, the taxonomic analyzer 230, functional analyzer 232 and integrated analyzer 234 may include program instructions for the processes described in FIG. 5, FIG. 8 and FIG. 10.

FIG. 3 is a flowchart of a method 300 for obtaining microbiome data for visualizing and analyzing using the system of FIG. 2, according to some embodiments.

Method 300 may begin by acquiring samples from an animal trial at step 302.

At step 304 DNA extraction and sequencing is performed.

At step 306 filter demultiplexed reads may be performed on the sequenced data for quality.

Quality control of sequencing may be performed on raw, demultiplexed reads to ensure proper removal of adapters and ensure appropriate length of sequences is maintained for downstream processing. Sequences are first examined using FastQC™ for an 11-dimension analysis of the incoming sequences. Sequences are then trimmed using CutAdapt™, Trimmomatic™, or any sequence filtering software. In some embodiments, trimming and filtering of reads can use a tool such as CutAdapt™ (described at DOI: https://doi.org/10.14806/ej.17.1.200). Cutadapt finds and removes adapter sequences, primers, poly-A tails and other types of unwanted sequence from high-throughput sequencing reads.

Host filtering can require alignment against a host genome if no custom functional database exists that has been pre-screened against all host DNA

The filtered reads can be further aligned against host DNA if no pre-filtered database exists downstream. This filtering may be done using Burrow-Wheeler Alignment tool (BWA) and removing reads or read-pairs that have mappings to the host genome.

At step 308, the filtered data is classified according to taxonomy and function.

Identifying taxonomy may be performed by using a one dimensional FastQ™ as inputs to Metaphlan2™.

Characterizing functions may use BWA against custom databases pre-filtered for host genes. In some embodiments, the metabolic network may be hand-curated against MetaCyc/KEGG/literature and can utilize several different annotations for matching functional data to reaction edges.

Taxonomy identification may be done on all forward reads that have passed the filtering and quality control step. To classify sequences, filtered forward reads may be fed into MetaPhlan2. Custom scripts can recompile this into an interactive visualization that has been re-associated with the sampling metadata. In some embodiments, this visualization is implemented using Microsoft Excel's PivotChart™ feature.

MetaPhlan2 is used because of its marker-gene approach for assigning taxonomy and the relative improvement in detection of more microorganisms.

Function classification in some embodiments may be performed using an alignment algorithm for the forward and reverse (if present) reads onto custom databases. A BWA tool may be used. These databases may be custom built around microbiome environments of interest and hand-curated to produce optimal results around understanding which genes are present. The present genes can then be mapped onto a custom metabolic network with prokaryotic specific pathways for interactive visualization using software such as, for example, Gephi.

The comparison of identifying function can be done for using the results obtained from the sequencing vendor, an internal mapping to a publicly available for purchase database, and/or a custom internal database.

At step 310, the classified data is prepared for interactive visualization. In some embodiments, as noted above the taxonomic classification from a tool such as, for example, Metaphlan2 can be recompiled using custom scripts into an interactive visualization that has been re-associated with the sampling metadata. In some embodiments, this visualization is implemented using Microsoft Excel's PivotChart feature.

The functional classification can be visualized by mapping the genes found to be present onto a custom metabolic network with prokaryotic specific pathways. The interactive visualization of the functional classification can be implemented using Gephi.

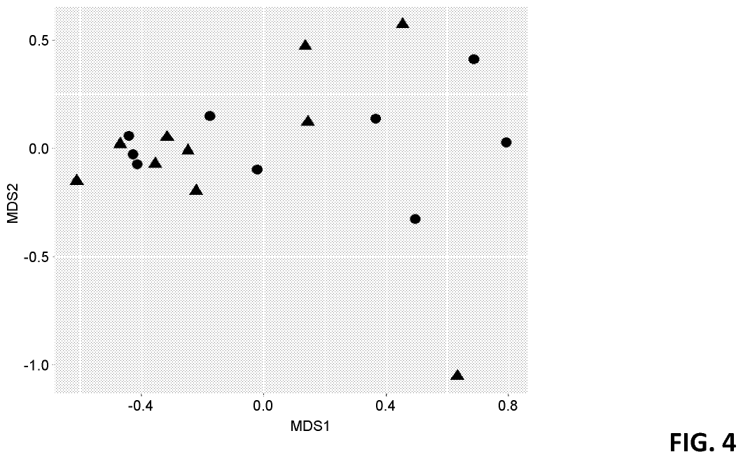

FIG. 4 is a visualization of taxonomic data from microbiome data shown on a display, according to a conventional system. Changes in microbial community are often visualized in conventional systems using non-metric multi-dimensional scaling (NMDS), e.g., Bray-Curtis distance, plots. However, often clear differences in a treatment are not apparent in these plots. FIG. 4 shows an NMDS plot of all samples within the two treatments. Each point represents the entire collection of a particular microbe within the sample and the different shapes represent the control (e.g. circles) and the treatment (e.g. triangles).

FIG. 5 is a flowchart of a process 500 for taxonomic visualizing and analyzing microbiome data using the system of FIG. 2, according to some embodiments. Process 500 may be performed by a computer system such as that shown in FIG. 2. As noted above in relation to FIG. 4, conventional techniques such as NMDS plots for visualizing changes in the microbial community often do not clearly show the differences in a treatment are not apparent. Process 500 provides substantial improvements in the changes that can be detected when evaluating the network structure of the microbial community.

After entering the process 500, at operation 502, the process accesses microbiome data from a selected sample and/or group. The microbiome data may be derived from a process such as that shown in FIG. 3.

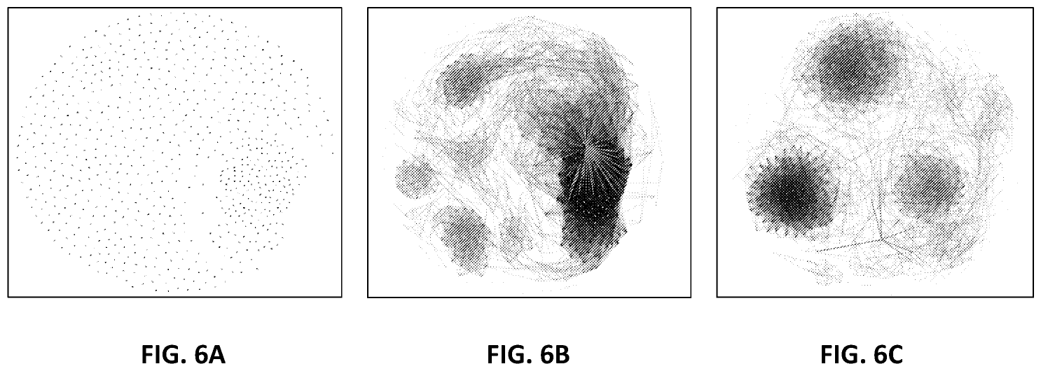

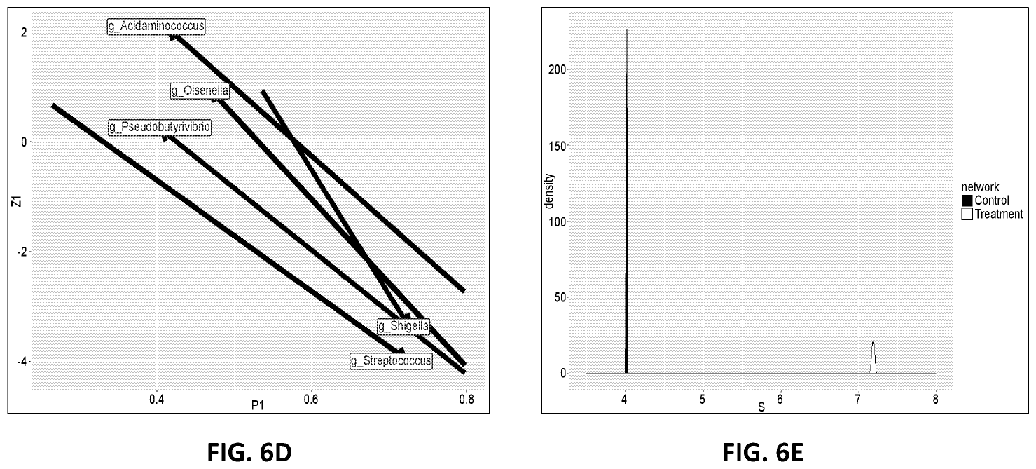

At operation 504, the data is analyzed to determine the set of nodes in the dynamic network to be generated. All organisms present in all samples may be identified in this operation. In this example, microbes and/or other OTU that are present in the microbiome data are to be represented as nodes in the dynamic network. These form the basic scaffold of the network. To construct the base network, each OTU serves as an independent node and no edges exist. A base network is shown in FIG. 6A. In an example implementation, a software such as Igraph™ (e.g., within C, R, or Python), may be used to generate an empty graph with the identified organisms as nodes.

At operation 506, the characteristics of the microbiome data to be represented in the edges of the dynamic network are determined. In this example, correlation between pairs of microbes is determined.

To construct the edges to complete the network, some example embodiments use a correlation measure such as, for example, Spearman Correlation data of each OTU to every other OTU. OTU data from each group may be separated and run through Spearman Correlations of each OTU to each other OTU. These Spearman Correlations may serve as the dynamic edges in the network, where under the control conditions, the Spearman Correlations may be considered to reflect the strength and association of each microbe to every other microbe for the control group of the host animal and under treated conditions, the strength and association of each microbe to every other microbe may be considered to be modulated by the treatment.

The creation of connections between these organisms can be done across samples, treatment groups, or other ways of arranging samples.

In example embodiments, connections can be created based on one or more of absence/presence, correlations, taxonomy, expected number of genes within the organisms, and shared gene functions within the organisms.

After the connections have been derived, they can be embedded into the dynamic network using a software tool such as, for example, an XML library (e.g., lxml in Python, xml or xml2 in R, etc.).

At operation 508, the dynamic network is generated for a selected sample and/or group. For example, the dynamic network for the control group and an initial timestamp may be generated. The dynamic network, in this embodiment, comprises of microbes represented in nodes, and the correlation between respective pairs of microbes represented in the edges.

As noted above, in some embodiments, connections other than correlation between organisms can be used as the edges. Connections can, but do not have to be, identified as the weight. Multiple ways of connecting the organisms can be embedded in the same network graph. For example, in some embodiments, the dynamic network can have both absence/presence and correlation of those two organisms represented in the edges.

In some embodiments, edges and/or nodes are associated with a variable that enable dynamic transitions. For example, such a variable may provide network visualization software to dynamically change the displayed network to clearly show differences in the network among the different conditions and/or samples of the microbiome. In some examples, one or more attributes are provided to nodes and/or edges to represent variables such as, for example, condition represented in the sample, and a timestamp or time sequence of the sample.

The network once it has been modified into the appropriate format and the edges and/or nodes identified as dynamic, can be visualized. A software such as Gephi™ may be configured to provide dynamicity to the network based on such attributes. In Gephi, the dynamic format is recognized as a ‘.gexf’ (Graphical Exchange Format) and a timeline allows for rapid visualization between time points where time points can be either time in the case of a longitudinal study, concentration or otherwise numerically associated or conditions where the timestamps are indicative of a treatment, condition, or otherwise categorical in nature.

At operation 510, the dynamic network is displayed. The display may be of the entire dynamic network or a part of the network.

At operation 512, network statistics may be calculated and displayed. The calculated network statistics may be based on the displayed portions of the dynamic network, thus enabling the user to be able to visually relate the statistics to the interactions among the microbes displayed as can be seen in the dynamic network.

In some embodiments, network statistics such as, for example, eigenvector centrality and betweenness centrality can be calculated. The former may be a measure of strongly an organism is connected to its own sub-community, and the latter may be a measure of how strongly an organism is connected to different sub-communities. These two measures, for example, can assist in the identification of keystone organisms.

At operation 514, the system may receive filtering control input to increase or decrease selected information shown in the dynamic network.

Filtering can be done to select for a narrow range of correlation strength (r=0.6). This filtering can be controlled interactively or automatically to reveal a characteristic structure and shape of the microbiome under control conditions (e.g. shown in FIG. 6B). Simply by allowing the dynamic network to change, the user can observe the new microbiome structure in swine that were treated (e.g. shown in FIG. 6C). These changes in the network structure are expected to change the potential flow of information and allow for understanding of keystone microbes. Using network statistics like eigenvector centrality and betweenness centrality the user can, in relation to the displayed network, identify keystone organisms.

In FIGS. 6B and 6C below, under control conditions (FIG. 6B), OTU630 has the largest eigenvector centrality at 0.85 while OTU539 has the largest betweenness centrality at 0.597. Changing to the treatment (FIG. 6C), it is OTU164 that has the maximum eigenvector centrality (1.0) and OTU147 with the maximum betweenness centrality (0.064). In the treatment condition, OTU630 has a much lower eigenvector centrality (0.004) and OTU539 has a much lower betweenness centrality (0.002).

At operation 516, interactive input or automated selection of a sample for which the dynamic network is to be displayed may be received. For example, the user may use the controls, such as controls 108 and/or 110 to control filtering and/or control the selection of the group and/or timestamp of the sample being currently displayed in the dynamic network.

In an example implementation, the microbiome data is derived from samples were collected from two groups of swine (one control and one treatment) by extracting DNA from the cecum after 30 days post-natal. These samples were sequenced to produce operational taxonomic units (OTU) by a process such as that described in relation to FIG. 3. FIG. 6 shows the use of the dynamic network for the sample.

FIG. 6 (FIGS. 6A, 6B, 6C, 6D, and 6E) show respective display screens during a visualization and analysis using the process of FIG. 5, according to some embodiments.

FIG. 6A shows a scaffold of all organisms that are expected to co-vary. No edges/connections have yet been included and therefore this network currently has no structure.

FIG. 6B illustrates the control condition. Organisms that were found to co-vary (Spearman correlation r≥0.6) had an edge drawn between them. These edges are what create the ball-like structures within the network.

FIG. 6C illustrates the treatment condition. Organisms that were found to co-vary (Spearman correlation r≥0.6) had an edge drawn between them. These edges are what create the ball-like structures within the network.

A visual comparison of FIGS. 6B and 6C illustrates that the network in FIG. 6C has a clearly different structure now that a treatment has been applied

FIG. 6D illustrates a screen showing the distribution of connectivity of organisms within the network that experienced the largest shifts (e.g., ZiPi distance). Increasing P1 shows an increase connectivity to multiple groups in the network and an increasing Z1 shows an increasing connectivity to an individual group.

FIG. 6E illustrates a screen by which the network structure can be evaluated for ‘small-worldedness’ by the factor S. An S>>1 indicates a small-world effect. It the figure, it can be seen that the strength of the small-world effect increases with treatment. This indicates that more organisms/nodes are tightly connected than before.

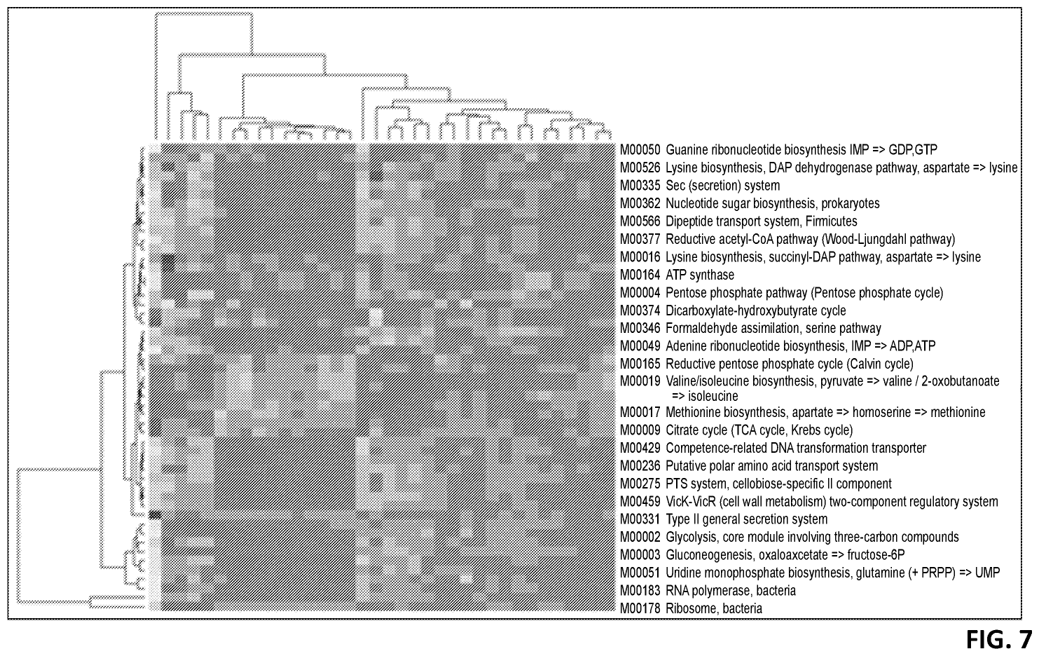

FIG. 7 is a visualization of functional data from microbiome data according to a conventional system. The heat map, shown in FIG. 7, shows the relative abundance of KEGG modules (i.e. modules defined by KEGG database) for all samples.

FIG. 8 is a flowchart of a process 800 for functional visualizing and analyzing microbiome data using the system of FIG. 2, according to some embodiments. Process 800 may be performed by a computer such as that shown in FIG. 2.

Typical metabolism analysis as in conventional systems collapses genes into unique pathways and looks at overall changes as shown in a heat map such as that FIG. 7. However, only a high level overview of what is going on from a functional level. In contrast to conventional techniques, process 800 provides a gene-level metabolic network where the nodes are metabolites and the edges are enzymatic reactions between the metabolites.

Although, different metabolic networks such as KEGG (www.genomejp/kegg) or GOMixer (http://www.raeslab.org/omixer/visualisation/map) exist as conventional systems, these merely form the scaffold and don't facilitate any in-depth analysis. These conventional metabolic networks are also static and don't allow for transitions between different environments, times, or treatments that may be part of an experiment.

After entering process 800, at operation 802, microbiome data may be accessed from one or more groups and for one or more time samples. The microbiome data may be derived from a process such as that shown in FIG. 3.

At operation 804, metabolites in the data may be determined. All or part of the reactions and associated metabolites of desire to evaluate for samples are identified in this operations. The identified metabolites form the basic scaffold of the network. An empty graph can be made with these identified metabolites by using a software such as, for example, igraph (within C, R, or python).

At operation 806, per sample and per group, counts of metabolites are determined.

Connections between these metabolites may be created by identifying the reaction(s) between different metabolites. Connections can be created by: literature defined reactions; or KEGG, MetaCyc, BRENDA, eggNOG, or other publicly available databases.

At operation 808, a dynamic network of nodes (metabolites) and edges (genes/proteins/enzymes) is generated.

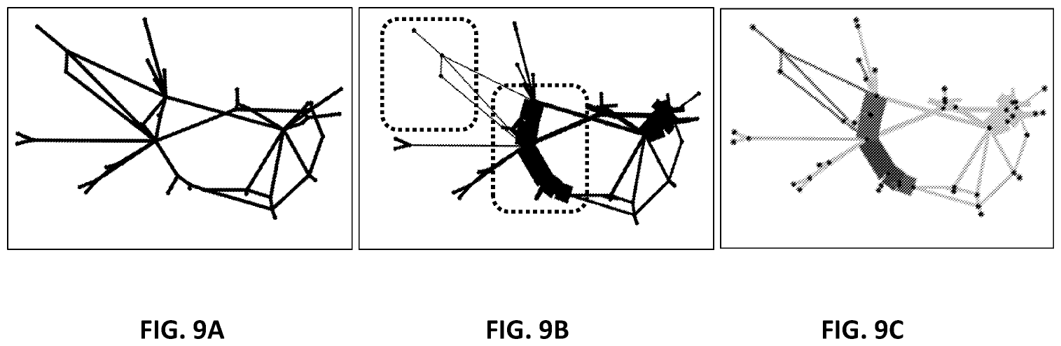

Next, the gene counts are matched against the edges of the network and the relative abundance of gene counts of each edge are normalized to our control such that the control edges all have a weight equal to one (FIG. 9A). By someone skilled in the field, the static network graph can be converted to dynamic and additional treatments can be added into the network.

At operation 810, display dynamic network for full/partial microbiome.

Once the connections have been derived, they can be embedded into a dynamic graph using an XML, library (e.g., lxml in Python, xml or xml2 in R, etc.). Metabolites function as nodes/vertices, and reactions/enzymes serve as the edges. Relative abundance, normalized abundance, or other numerical representations of the enzymes can, but do not have to be, identified as the weight.

Multiple ways of connecting the reactions can be embedded in the same graph. Can have both known KEGG reactions and MetaCyc reactions of those two organisms. Can have both relative and normalized abundance.

Importantly, edges or nodes are associated with a second variable that is recognized by the graphing visualization software that is dynamic. In the Gephi (V 0.9.2) visualization tool, timestamp is a way of doing so.

The graph once it has been modified into the appropriate format and the edges and/or nodes identified as dynamic, can be visualized. In gephi, the dynamic format is recognized as a ‘.gexf’ (Graphical Exchange Format) and a timeline allows for rapid visualization between time points where time points can be either time in the case of a longitudinal study, concentration or otherwise numerically associated or conditions where the timestamps are indicative of a treatment, condition, or otherwise categorical in nature.

Additionally, the reactions in the network can be overlaid with a complete set of statistics, notes, etc. that can allow visualization by multiple parameters

At operation 812, calculate network statistics/update display. The calculated network statistics may be based on the displayed portions of the dynamic network, thus enabling the user to be able to visually relate the statistics to the interactions among the metabolites displayed as can be seen in the dynamic network.

At operation 814, receive filtering control input, and at operation 816, receive group/sample control input. For example, the user may use the controls, such as controls 108 and/or 110 to control filtering and/or control the selection of the group and/or timestamp of the sample being currently displayed in the dynamic network.

According to some embodiments, samples were collected from two groups of swine (one control and one treatment) by extracting DNA from the cecum after 21 days post-weaning. These samples were sequenced using a shotgun metagenomic approach. The sequence reads were aligned to a database of annotated gene sequences to produce a count table of genes for each sample from both groups.

FIG. 9 shows the use of the dynamic network for the samples. Looking at the treatment group, there is a clear increase in certain enzyme reactions and decrease in others relative to the control group shown in FIG. 9A. A decrease in edges 197, 166, 167, and 168 between nodes/metabolites 0, 48, 75, 76, and 90 (identified top left; FIG. 9B) can be observed. There is an increase in certain edges associated with respective nodes/metabolites (identified in the center; FIG. 9B). Running statistics, these can be embedded in this same graph and p-values (probability) of the treatment relative to the control can be visualized (FIG. 9C). This shows that those decreases and increases are indeed significant. Relating what is known about the treatment and the animals within the study, the user can associate these pathways as protagonistic or antagonistic to the host animals.

FIG. 9 (FIGS. 9A, 9B, and 9C) show respective display screens during a visualization and analysis using the process of FIG. 8, according to some embodiments.

FIG. 9A illustrates a network that was generated using information from a public database. The nodes are metabolites, and connections/edges are genes/enzymes or known reactions. The network shown is a control condition. The width of each reaction is normalized to a control condition.

In FIG. 9B, edges show a change in abundance relative to a control condition (shown in FIG. 9A), where reactions in the upper left (e.g. 902) see a decrease, whereas reactions in the middle (e.g. 904) see a large increase.

In FIG. 9C, information relating to the statistical significance of the change in abundance of the reactions (e.g. in 904 shown in FIG. 9B) between metabolites is shown overlaid onto the network. This shows the statistical significance of the change in the reaction relative to the control as a function of color (e.g., black to white) or other aspect.

FIG. 10 is a flowchart of a process for integrated functional and taxonomic visualizing and analyzing microbiome data using the system of FIG. 2, according to some embodiments. Taxonomic visualization was described in relation to FIGS. 5 and 6 above, and functional visualizing was described in FIGS. 8 and 9 above. The process may be performed by a computer such as that shown in FIG. 2.

The integrated taxonomic and metabolic analysis facilitated by example embodiments provide for diagnostic uses such as studying the effects of different dosages of a drug over time between a control group and a treatment group.

After entering operation 1000, at operation 1002, all or part of the reactions and associated metabolites of desire to evaluate are identified for samples.

At operation 1004, a scaffold for the network is generated from the identified metabolites and reactions. Using a software such as, for example, igraph (within C, R, or python), an empty graph can be made with these metabolites.

Connections may be created between the identified metabolites by identifying the reaction(s) between different metabolites. Connections can be created by: literature defined reactions; and/or KEGG, MetaCyc, BRENDA, eggNOG, or other publicly available databases.

The dynamic network is generated from the identified metabolites and reactions.

For every organism (or hierarchical subset of organisms, i.e. taxonomy) that the user requires to be separated out, the reaction network created in the above steps can be duplicated for as many organisms (or relevant subsets) with relevant identification (organism name, taxonomy, etc.).

Once the number of networks and reactions have been identified and built, they can be embedded into a dynamic network using a software such as, for example, an XML library (e.g., lxml in Python, xml or xml2 in R, etc.).

Metabolites function as nodes/vertices in the dynamic network. Reactions/genes/enzymes serve as the edges and are also identified by relevant taxonomy (or other grouping information).

Relative abundance, normalized abundance, or other numerical representations of the genes/enzymes can, but do not have to be, identified as the weight.

Multiple ways of connecting the reactions can be embedded in the same graph. Edges can have both known KEGG reactions and MetaCyc reactions of those two organisms. Also, edges can have both relative and normalized abundance.

Importantly, edges or nodes are associated with a second variable that is recognized by the graphing visualization software that is dynamic. In the Gephi (V 0.9.2) visualization tool, timestamp is a way of doing so.

The network once it has been modified into the appropriate format and the edges and/or nodes identified as dynamic, can be visualized.

In gephi, the dynamic format is recognized as a ‘.gexf’ (Graphical Exchange Format) and a timeline allows for rapid visualization between time points where time points can be either time in the case of a longitudinal study, concentration or otherwise numerically associated or conditions where the timestamps are indicative of a treatment, condition, or otherwise categorical in nature.

Additionally, the reactions in the network can be overlaid with a complete set of statistics, notes, etc. that can allow visualization by multiple parameters.

This will dynamically change all edges/reactions for every group of organism, taxonomic clusters (or other grouping).

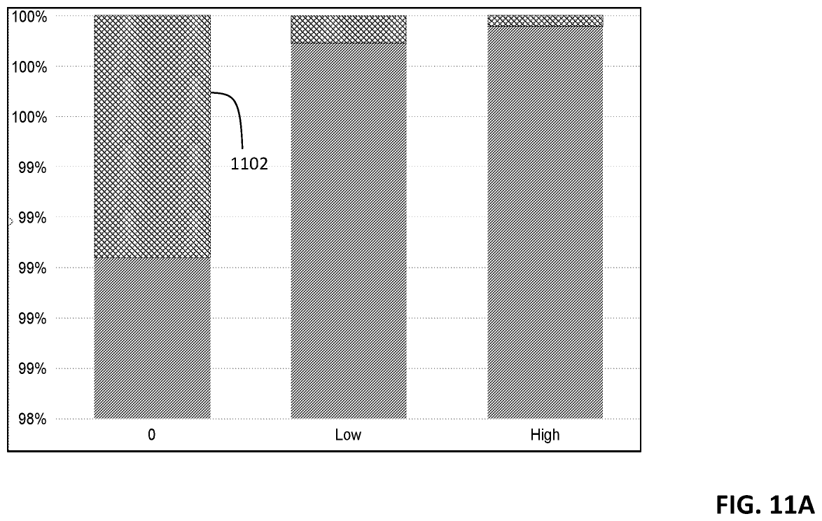

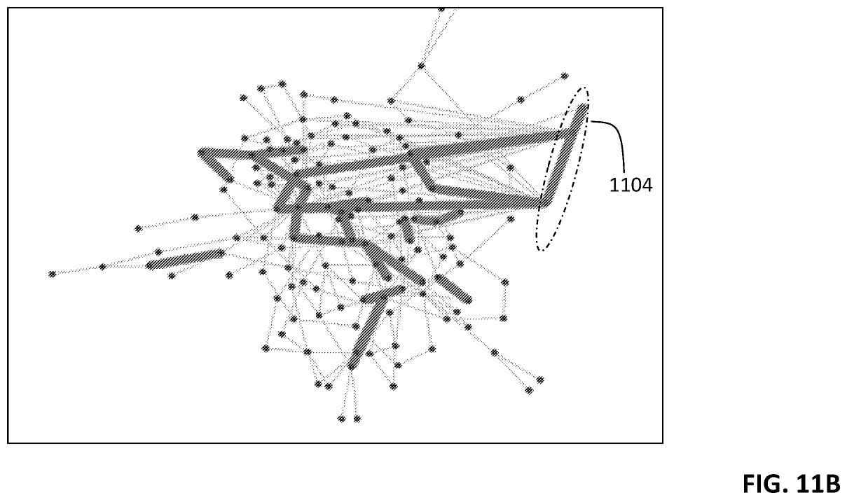

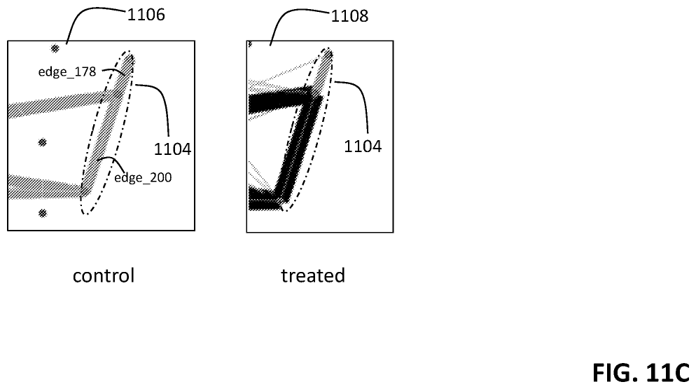

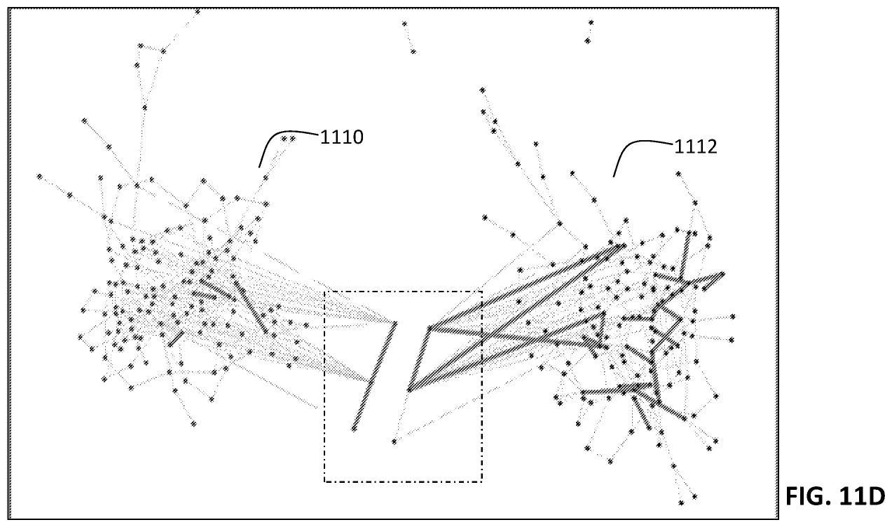

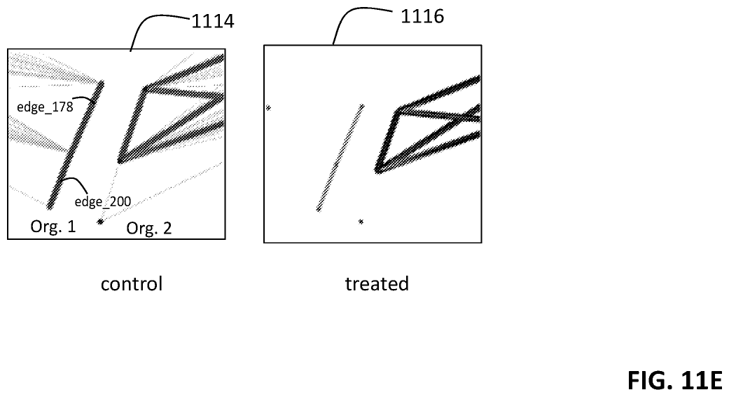

The initial functional network visualized under control conditions showed a rather sparse network (FIG. 11B).

At operation 1008, the user may choose to focus on one or more particular reactions.

For example, the user may choose one or more metabolites (e.g., 1104) for further investigation. Focusing on the effect of the added enzyme, changes in the pathway can be visualized (FIG. 11B).

At operation 1010, the user may choose to view the effect seen at operation 1008, which is on a supra organism, as it affects each individual organism in the supra organism. This effect for the two selected organisms is shown in FIG. 11C.

At operation 1012, an area of interest for further investigation of the selected organism may be selected. As shown in FIG. 11D, the selected organism (in this particular example, organism 1 and organism 2) can be viewed side by side.

At operation 1016, the user may select to view the individual organisms for different timestamps, such as, for example, at different treatment stages as shown for example in FIG. 11E.

An implementation of process 1000, and screens displayed by a computer system such as that shown in FIG. 2 are shown in FIG. 11. In a trial, two groups of swine were subjected to two different doses (a high dose and a low dose) of an enzyme. Samples were also collected from a third group of animals, as a control group. These samples were sequenced using a shotgun metagenome approach and the results were interpreted using MetaPhlan2 for taxonomy and KEGG for function.

This enzyme appeared to have an effect on the structure of the community by impacting two selected microorganisms differently (as shown in FIG. 11A: 0—Control, Low Dose, High Dose). Organism 1 increased while organism 2 decreased.

Using the reactions between selected nodes (e.g. 1104), one enzymatic reaction is characterized as increasing (edge 200) and one enzymatic reaction is characterized as decreasing (edge 178). Going back to the database from which the identified microbial genes were identified, organism information may be coupled to the functional annotations to understand how the shift in metabolism was being derived from these organisms. The two organisms metabolic network were first visualized (FIG. 11D) with the nodes identified in FIG. 11B placed near each other. It is interesting to note that one of the two enzymatic reactions being focused on does not exist in one of the two organisms suggesting that the entire contribution of this reaction comes from an individual organism. Visualizing the change in the enzymatic reactions of the same edges but from two different organisms, we can explain the changes in FIG. 11B more thoroughly. The decrease in edge 178 is derived from a decrease in organism 2. While edge 200 does increase overall (FIG. 11B), organism 1 contributes to an increase while organism 2 decreases at that edge. The edge that is decreasing is likely not conferring a benefit to the organism and therefore does not provide organism 2 any fitness advantage. Adding in the metabolite at node 80 may increase the fitness benefit of edge 178 and increase this organism. This shows how dynamic visualization of metabolic networks integrated with taxonomic information can allow for the development of molecules to selectively manipulate different organisms within the overall population.

FIG. 11 (FIGS. 11A, 11B, 11C, 11D and 11E) show respective display screens during a visualization and analysis using the process of FIG. 10, according to some embodiments.

FIG. 11A illustrates, focusing on two organisms (for simplicity), that it can be visually shown that there is a large change in the relative abundance due to the dose of the applied treatment. Going from left to right, the control, low dosage and high dosage groups are indicated. It can be seen that the organism corresponding to 1102 has decreased when the dosage is increased, whereas the other organism has increased.

FIG. 11B illustrates, considering these two organisms as operating together (sometimes referred to as a supra-organism), the functional analysis where the detected genes/enzymes are shown in grey as edges and the potential metabolites are shown in black as dots/nodes. Three metabolites 1104 were selected for further focus.

FIG. 11C illustrates, focusing on just these couple of selected metabolites, that it appears the reactions, when treated, between two of them are getting thicker (1108) relative to the control (1106). The individual effect of each organism is not known in this situation.

FIG. 11D illustrates, using the network created for each organism, that the genes can be mapped accordingly. Organism 1, represented in 1110, and organism 2, represented in 1112.

FIG. 11E illustrates, under control conditions (1114), that organism 1 is shown to have both genes catalyzing the reactions between all three metabolites, where organism 2 only has one gene. Under high dose of enzymes (1116), organism 2 genes are seen to increase but there is a general decrease in organism 1.

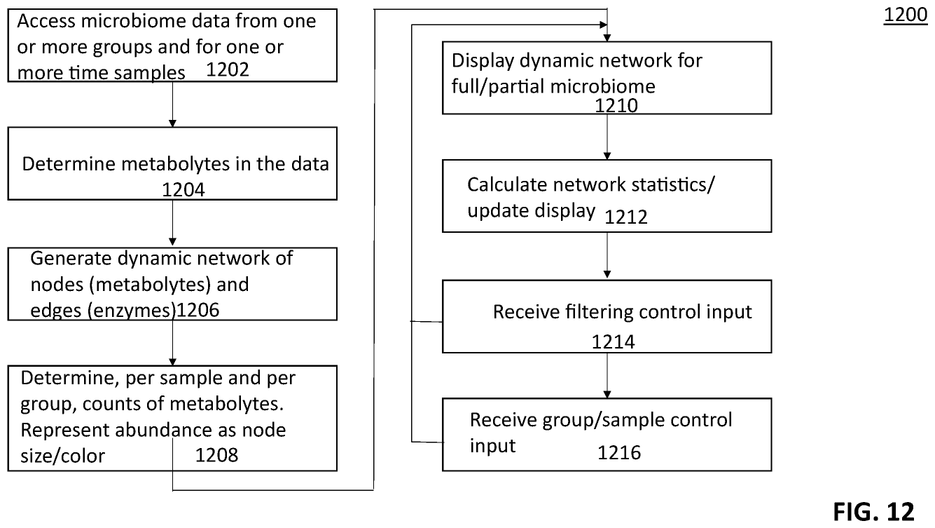

FIG. 12 is a flowchart of a process 1200 for identifying genes and/or metabolites for diagnostics relative to control condition using the system of FIG. 2, according to some embodiments. Process 1200 can be performed by the computer system 200 to identify changes in microbiome and potential metabolites, which provide an early warning diagnostic that could facilitate intervention.

After entering process 1200, at operation 1202 microbiome data may be accessed from one or more groups and for one or more time samples.

At operation 1204, all or part of the reactions and associated metabolites of desire to evaluate for samples are identified. These form the basic scaffold of the network. A software such as, for example, igraph (within C, R, or python), can be used to create an empty graph made with the identified metabolites.

Connections between these metabolites may be created by identifying the reaction(s) between different metabolites. Connections can be created by: literature defined reactions; and/or KEGG, MetaCyc, BRENDA, eggNOG, or other publicly available databases.

At operation 1206, once the connections have been derived, they can be embedded into a dynamic graph using an XML library (e.g., lxml in Python, xml or xml2 in R, etc.).

In the dynamic network, metabolites function as nodes/vertices.

At operation 1208, relative abundance, normalized abundance, or other numerical representations of the metabolites can, but do not have to be, identified as the node size/color.

One of several ways of identifying the metabolites can serve as the information for creating node sizes. The technique used may be one of metabolic flux analysis, HPLC, NMR, etc.

One or both relative and normalized abundance can be embedded.

Reactions/enzymes serve as the edges of the dynamic network.

Relative abundance, normalized abundance, or other numerical representations of the enzymes can, but do not have to be, identified as the weight for edges.

Multiple ways of connecting the reactions can be embedded in the same graph. For example, known KEGG reactions and MetaCyc reactions of those two organisms can both be embedded. One or both relative and normalized abundance can be embedded.

Importantly, edges or nodes are associated with a second variable that is recognized by the graphing visualization software that is dynamic. Timestamp is a way of doing so.

At operation 1210, the network once it has been modified into the appropriate format and the edges and/or nodes identified as dynamic, can be visualized.

In gephi, the dynamic format is recognized as a ‘.gexf’ (Graphical Exchange Format) and a timeline allows for rapid visualization between time points where time points can be either time in the case of a longitudinal study, concentration or otherwise numerically associated or conditions where the timestamps are indicative of a treatment, condition, or otherwise categorical in nature.

Additionally, the reactions in the network can be overlaid with a complete set of statistics, notes, etc. that can allow visualization by multiple parameters.

Operations 1212-1216 provide for the user to interact with the dynamic network in manners similar to that described in relation to other processes above.

In an implementation, process 1200 was used to identify changes in microbiome and potential metabolites in post-weaning diarrhea in animals. Post-weaning diarrhea is an affliction that affects piglets and can result in increases in mortality of the piglets. Identifying changes in microbiome and potential metabolites can create an early warning diagnostic that could facilitate intervention. In a study of post-weaning diarrhea, a cohort of piglets was tracked over 20 days and samples were taken for microbiome sequencing, metabolite extraction, and a fecal score was recorded. A fecal score can be an integer between 0 (no diarrhea) to 3 (severe diarrhea) and these results are usually recorded currently by human observation. In an example embodiment, this data can be used after sequencing.

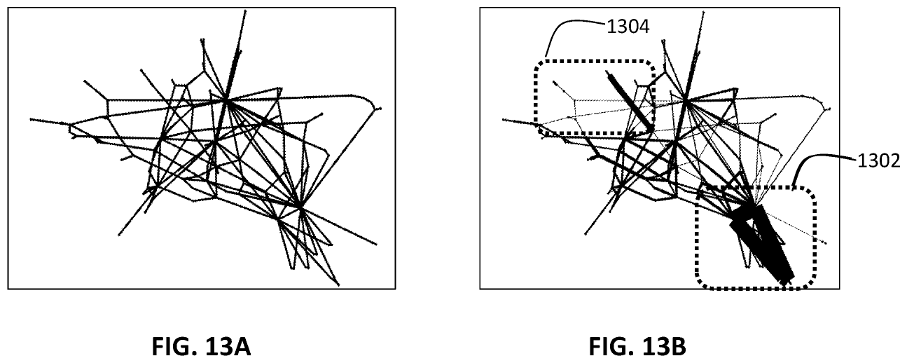

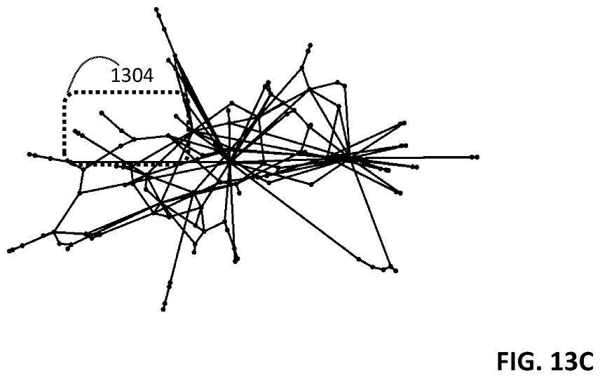

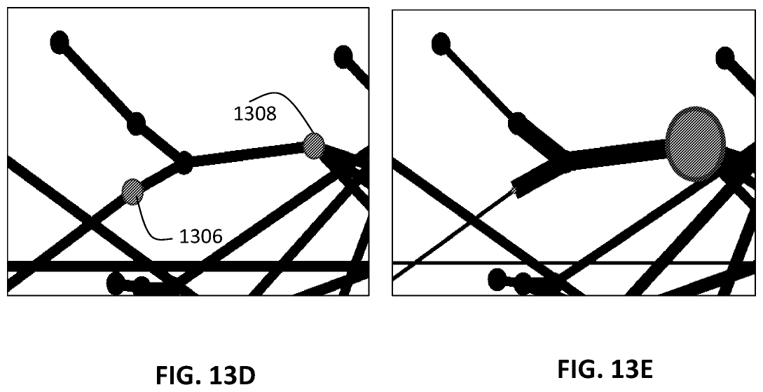

After sequencing the microbiome and identifying genes present in the pathways, changes could be normalized to a fecal score of 0 (no diarrhea), as shown in FIG. 13A, to look for changes. Examining the network at a fecal score of 3, a large shift in pathways can be seen to be both increasing (FIG. 13B; bottom right 1302) and decreasing (FIG. 13B; top left 1302). The increasing enzymatic reactions, edges 217 and 220, can be further examined as a biomarker associated with increasing fecal score. On the inverse, examining decreased pathways can identify bottlenecks that can become potential treatment goals (probiotic containing missing/decreased pathways), or identify upstream metabolic markers that may serve as another diagnostic target. To prove this could be used to identify the metabolic markers and how the metabolic network is directly tied to these metabolites, metabolites were integrated into the metabolic network. Focusing on an area where reactions are decreasing (FIG. 13C, 1304), it would be expected that an accumulation of a metabolite would occur. With measured metabolites shown as 1306 and 1308 in FIG. 13D, one metabolite can be visually described as decreasing that is also adjacent to decreased reactions (FIG. 13E). Upstream of this metabolite where enzymatic reactions are still present, there is an accumulation of a metabolite. This sharp change in abundance of enzymatic reactions would be known as a bottleneck (FIGS. 13B and 13D), where without measurement of metabolite data (FIG. 13B), can generate hypothetical metabolic markers as targets. The next step incorporating measurements of metabolites (FIG. 13E) can solidify the user's understanding of where the accumulation is actually occurring.

FIG. 13 (FIGS. 13A, 13B, 13C, 13D and 13E) show respective display screens during a visualization and analysis using the process of FIG. 12, according to some embodiments.

FIG. 13A illustrates the base metabolic network for the desired state (e.g., no diarrhea). All detected edges here are normalized to this state.

FIG. 13B illustrates that, upon shifting to a worsening condition, it can be visualized as the decrease in pathways, upper left box 1304, and increase in other reactions, lower right box 1302.

FIG. 13C illustrates that, metabolite data if available, can be embedded to increase the information derived from the dynamic metabolic network (not all metabolites are measured). This is again the control state of the network (no diarrhea). The area where a decrease was seen in some reactions is highlighted in the box 1304 in the figure.

FIGS. 13D and 13E illustrate a zoomed in on the box from FIG. 13C, the control case is shown with measured metabolites shown in 1306 and 1308 in FIG. 13D. Upon quickly visualizing the dynamic network under severe diarrhea conditions as seen in FIG. 13E, the change in abundance of the metabolite upstream of the missing enzymes is seen. The decrease in the metabolite downstream, and right at the inflection point in gene abundance, suggests both a direction of metabolite flow and what genes are important for the accumulation and dissipation of the increasing metabolite.

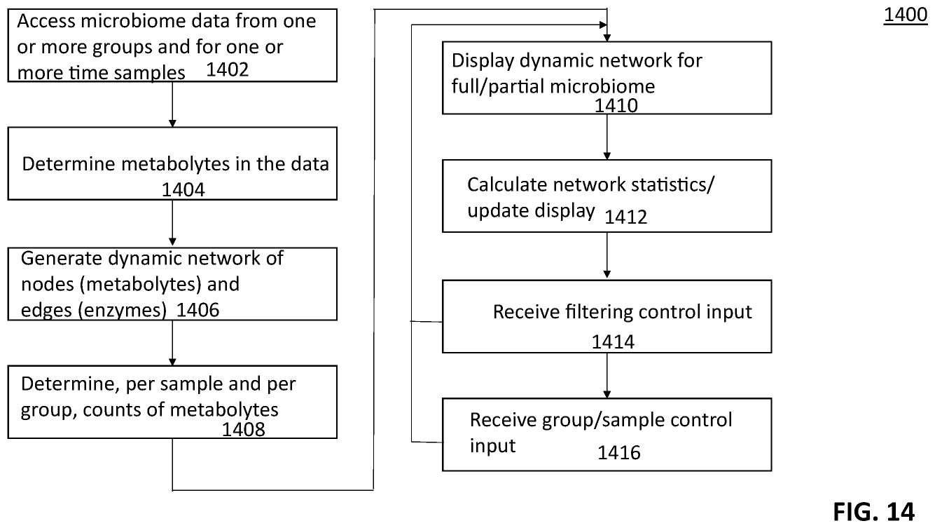

FIG. 14 is a flowchart of a process 1400 for using the system of FIG. 2 to develop internal standards to monitor fluctuations in microbiome, according to some embodiments.

After entering process 1400, at operation 1402 microbiome data may be accessed from one or more groups and for one or more time samples.

At operation 1404, all or part of the reactions and associated metabolites of desire to evaluate for samples are identified. These form the basic scaffold of the network. In igraph (within C, R, or python), an empty graph can be made with these metabolites.

Connections can be created between these metabolites by identifying the reaction(s) between different metabolites. Connections can be created by: literature defined reactions; and/or KEGG, MetaCyc, BRENDA, eggNOG, or other publicly available databases.

At operation 1406, once the connections have been derived, they can be embedded into a dynamic graph using an XML library (e.g., lxml in Python, xml or xml2 in R, etc.).

Metabolites function as nodes/vertices of the dynamic network. Reactions/enzymes serve as the edges. Relative abundance, normalized abundance, or other numerical representations of the enzymes can, but do not have to be, identified as the weight of edges.

Multiple ways of connecting the reactions can be embedded in the same graph. For example, the network can have both known KEGG reactions and MetaCyc reactions of those two organisms. One or both relative and normalized abundance can be represented.

Importantly, edges or nodes are associated with a second variable that is recognized by the graphing visualization software that is dynamic. Timestamp is a way of doing so.

At operation 1410, the graph once it has been modified into the appropriate format and the edges and/or nodes identified as dynamic, can be visualized.

In gephi, the dynamic format is recognized as a ‘.gexf’ (Graphical Exchange Format) and a timeline allows for rapid visualization between time points where time points can be either time in the case of a longitudinal study, concentration or otherwise numerically associated or conditions where the timestamps are indicative of a treatment, condition, or otherwise categorical in nature.

Additionally, the reactions in the network can be overlaid with a complete set of statistics, notes, etc. that can allow visualization by multiple parameters

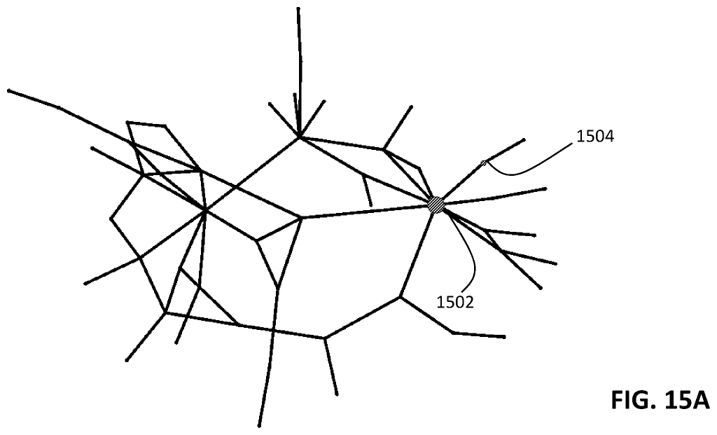

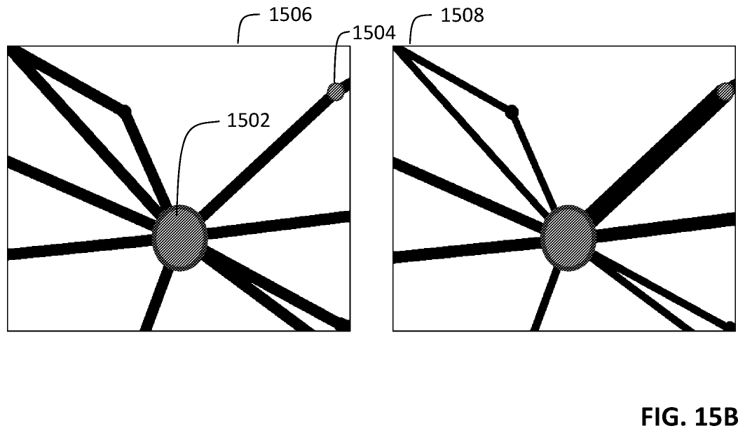

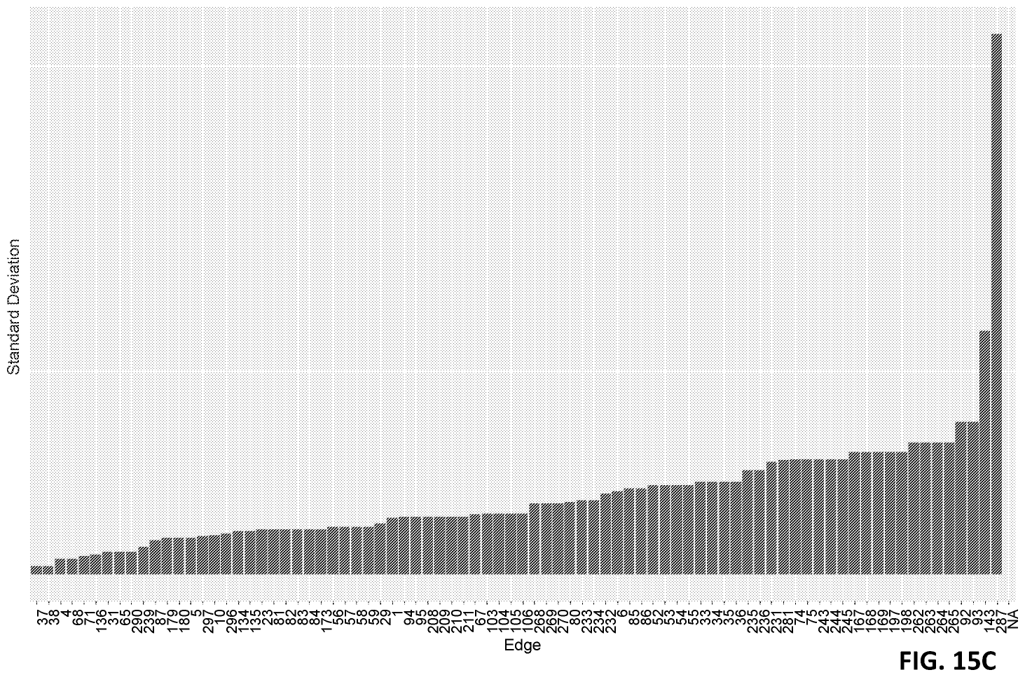

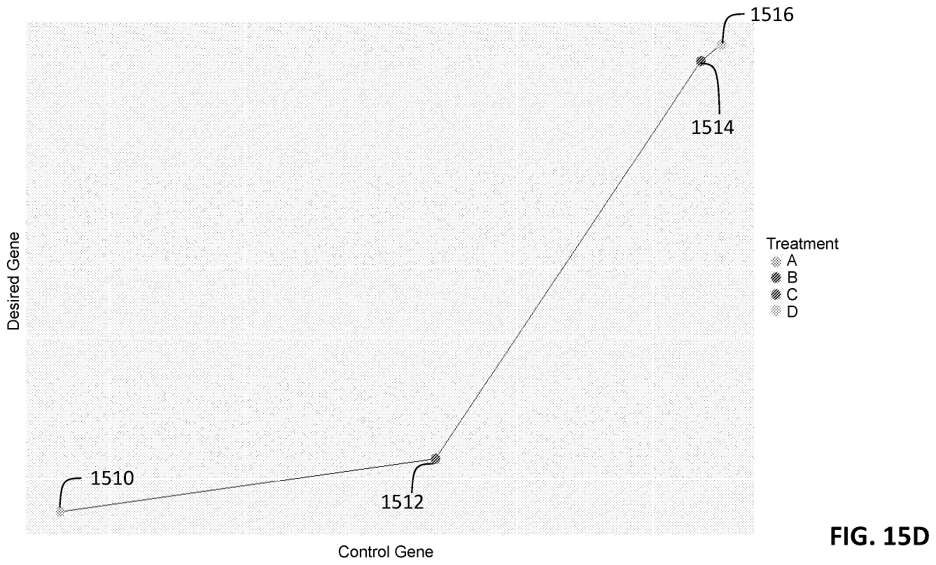

At operations 1412-1416, the user can interact with the dynamic network as, for example, described in relation to FIG. 15. A motif(s) can be rapidly identified by examining the visual changes in the graph to establish treatment and/or time effects.

Once edges to monitor are selected, identification of control edges can be selected by their low variance, standard deviation, or other metric to identify the variability.

The internal standard can then be compared to the gene(s) of interest to develop a diagnostic.

In an example implementation, samples were collected from four groups of swine (one control and three treatments) by extracting DNA from the cecum after 30 days post-natal. These samples were sequenced to produce operational taxonomic units (OTU's). These samples were sequenced using a shotgun metagenomic approach. Reads were aligned to a database of annotated gene sequences to produce a count table of genes for each sample from all groups. A gene-level metabolic network was constructed where the nodes are metabolites and the edges are enzymatic reactions between the metabolites.

Next, the gene counts are matched against the edges of our network and the relative abundance of gene counts of each edge are normalized to our control such that our control edges will all have a weight equal to one (FIG. 15A). By someone skilled in the field, the static network graph can be converted to dynamic and additional treatments can be added into the network. In this experiment, a desired phenotype was observed that was correlated with a change in a specific gene (FIG. 15B). Observing this change relative to the control, there is a need to develop an internal standard in the future case where every animal is being treated to monitor the effectiveness of the treatment without running a control group of animals. Characteristics of a good control gene/reaction/enzyme to establish as an internal standard is a gene that is relatively constant and does not fluctuate. All edges/genes/reactions were examined for their standard deviation (FIG. 15C). Using an edge with the lowest standard deviation, the changes in the desired gene can be more efficiently monitored internal to a single treatment (FIG. 15D).

FIG. 15 (FIGS. 15A, 15B, 15C and 15D) show respective display screens during a visualization and analysis using the process of FIG. 14, according to some embodiments.

FIG. 15A illustrates a metabolic network established in a control state. A metabolite of interest 1502 is highlighted with an increased size of the node/metabolite. An adjacent node 1504 is indicated for frame of reference in FIG. 15B.

FIG. 15B illustrates that compared to control conditions 1506, there is an edge increase due to a specific treatment 1508 that has a positive effect on the host animal. This gene/enzyme/reaction may therefore deemed important for determining a diagnostic.

FIG. 15C illustrates that to identify an internal control gene, selection of a reaction edge is important. Criteria generally would be considered to have low variability but can have other selection terms (such as a competing pathway(s), reaction(s), etc.)

FIG. 15D illustrates that, using an internal edge as a comparison, treatments 1512 and 1516 can be seen to increase the desired gene/reaction observed in FIG. 15B, 1508.

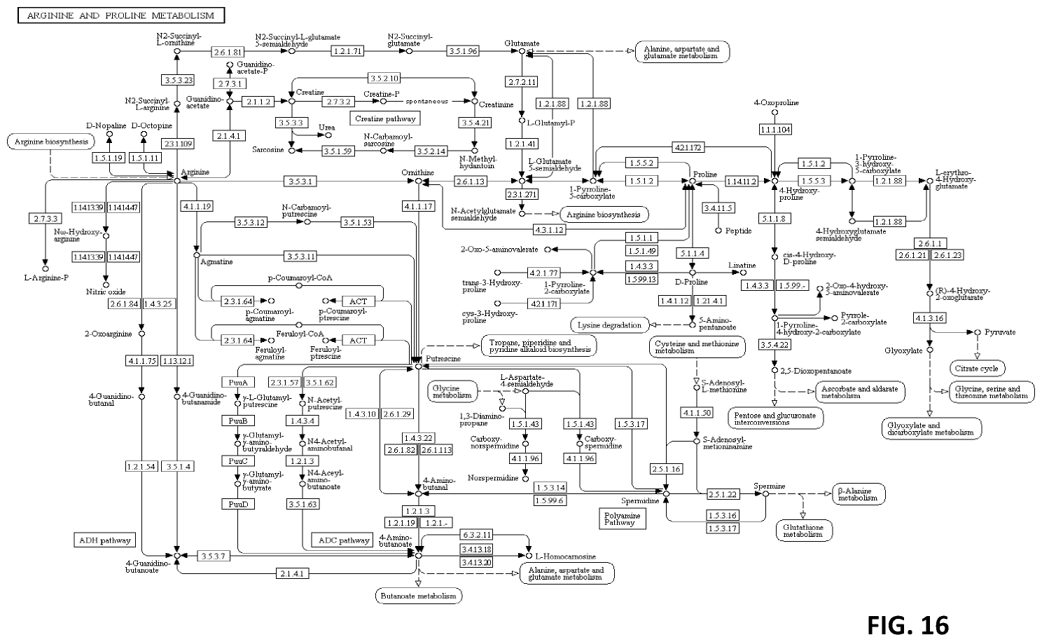

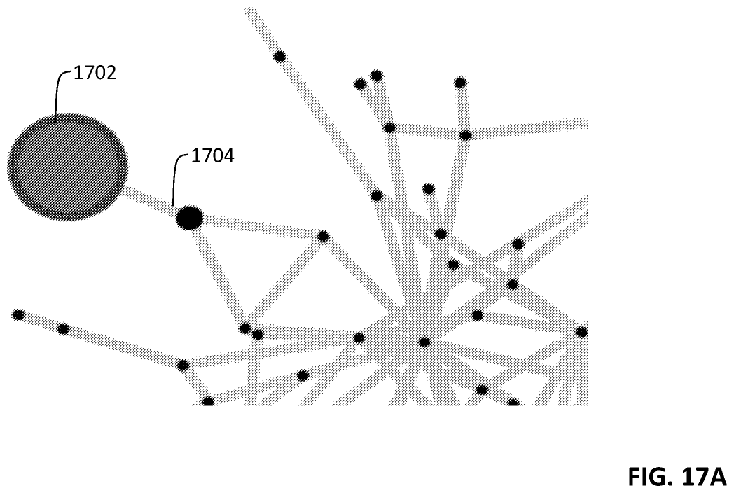

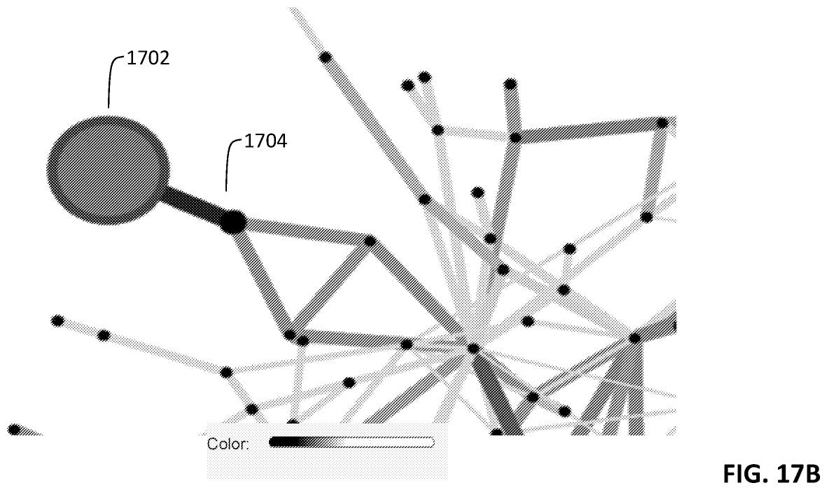

In another embodiment, discussed below in relation to FIGS. 16-17, host response identified targets may be used to infer microbiota produced signaling metabolites or pathway shared intermediates.

Molecules produced by the microbiome upon application of exogenous products majorly impact gene expression in host intestinal cells. It is known by people skilled in the art how to identify gene expression modification within eukaryotic cells. Some embodiments provide the capability to use the eukaryotic fluctuating internal host targets to identify dynamic expression of microbiota metabolites.

These microbiota metabolites identified by the dynamic microbiota model can either compensate for fluctuating intermediates within the host cell, after uptake (direct effect on host target) (targeted application of the dynamic microbiota model), or induce indirectly a metabolic answer/response from the host (ligand to receptor classical cascade signaling) (untargeted approach of the dynamic microbiota model).

Connections between the host and microbiome can be made, but is not limited to, by correlating microbiome pathways to host pathways based on a target node. Establish correlation with fluctuating molecules pattern based on knowledge-driven targets. Pathways with significant changes on both the host and microbiome side may indicate active communication.

The dynamic network can embed host responses and/or direct connections to metabolites to get a view of the strength and how the microbiome is connecting to the host. The host response to putrescine is represented by the largest red node and the molecule putrescine is represented by the large black connected node.

The dynamic network can allow for a multi-dimensional view into the microbiome-host connection. The width of the lines between nodes is the gene abundance relative to the control, and the color gradient represents the q-value (lower is more significant). This shows in the treated case that the microbiome has significant connections to putrescine and this is reflected in the connection to the host.

In the examples described herein, for purposes of explanation and non-limitation, specific details are set forth, such as particular nodes, functional entities, techniques, protocols, standards, etc. in order to provide an understanding of the described technology. It will be apparent to one skilled in the art that other embodiments may be practiced apart from the specific details described below. In other instances, detailed descriptions of well-known methods, devices, techniques, etc. are omitted so as not to obscure the description with unnecessary detail. Individual function blocks are shown in the figures. Those skilled in the art will appreciate that the functions of those blocks may be implemented using individual hardware circuits, using software programs and data in conjunction with a suitably programmed microprocessor or general purpose computer, using applications specific integrated circuitry (ASIC), and/or using one or more digital signal processors (DSPs). The software program instructions and data may be stored on computer-readable storage medium and when the instructions are executed by a computer or other suitable processor control, the computer or processor performs the functions. Although databases may be depicted herein as tables, other formats (including relational databases, object-based models, and/or distributed databases) may be used to store and manipulate data.

Although process steps, algorithms or the like may be described or claimed in a particular sequential order, such processes may be configured to work in different orders. In other words, any sequence or order of steps that may be explicitly described or claimed does not necessarily indicate a requirement that the steps be performed in that order. The steps of processes described herein may be performed in any order possible. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the technology, and does not imply that the illustrated process is preferred.

Processors, memory, network interfaces, I/O interfaces, and displays noted above are, or includes, hardware devices (for example, electronic circuits or combinations of circuits) that are configured to perform various different functions for a computing device, such as computer.

In some embodiments, each or any of the processors 1204 is or includes, for example, a single- or multi-core processor, a microprocessor (e.g., which may be referred to as a central processing unit or CPU), a digital signal processor (DSP), a microprocessor in association with a DSP core, an Application Specific Integrated Circuit (ASIC), a Field Programmable Gate Array (FPGA) circuit, or a system-on-a-chip (SOC) (e.g., an integrated circuit that includes a CPU and other hardware components such as memory, networking interfaces, and the like). And/or, in some embodiments, each or any of the processors 1204 uses an instruction set architecture such as x86 or Advanced RISC Machine (ARM).

In some embodiments, each or any of the memory devices is or includes a random access memory (RAM) (such as a Dynamic RAM (DRAM) or Static RAM (SRAM)), a flash memory (based on, e.g., NAND or NOR technology), a hard disk, a magneto-optical medium, an optical medium, cache memory, a register (e.g., that holds instructions), or other type of device that performs the volatile or non-volatile storage of data and/or instructions (e.g., software that is executed on or by processors). Memory devices are examples of non-volatile computer-readable storage media.

In some embodiments, each or any of the network interface devices includes one or more circuits (such as a baseband processor and/or a wired or wireless transceiver), and implements layer one, layer two, and/or higher layers for one or more wired communications technologies (such as Ethernet (IEEE 802.3) and/or wireless communications technologies (such as Bluetooth, WiFi (IEEE 802.11), GSM, CDMA2000, UMTS, LTE, LTE-Advanced (LTE-A), and/or other short-range, mid-range, and/or long-range wireless communications technologies). Transceivers may comprise circuitry for a transmitter and a receiver. The transmitter and receiver may share a common housing and may share some or all of the circuitry in the housing to perform transmission and reception. In some embodiments, the transmitter and receiver of a transceiver may not share any common circuitry and/or may be in the same or separate housings.

In some embodiments, each or any of the display interfaces in IO interfaces is or includes one or more circuits that receive data from the processors, generate (e.g., via a discrete GPU, an integrated GPU, a CPU executing graphical processing, or the like) corresponding image data based on the received data, and/or output (e.g., a High-Definition Multimedia Interface (HDMI), a DisplayPort Interface, a Video Graphics Array (VGA) interface, a Digital Video Interface (DVI), or the like), the generated image data to the display device, which displays the image data. Alternatively or additionally, in some embodiments, each or any of the display interfaces is or includes, for example, a video card, video adapter, or graphics processing unit (GPU).

In some embodiments, each or any of the user input adapters in I/O interfaces is or includes one or more circuits that receive and process user input data from one or more user input devices that are included in, attached to, or otherwise in communication with the computing devices, and that output data based on the received input data to the processors. Alternatively or additionally, in some embodiments each or any of the user input adapters is or includes, for example, a PS/2 interface, a USB interface, a touchscreen controller, or the like; and/or the user input adapters facilitates input from user input devices such as, for example, a keyboard, mouse, trackpad, touchscreen, etc.

Various forms of computer readable media/transmissions may be involved in carrying data (e.g., sequences of instructions) to a processor. For example, data may be (i) delivered from a memory to a processor; (ii) carried over any type of transmission medium (e.g., wire, wireless, optical, etc.); (iii) formatted and/or transmitted according to numerous formats, standards or protocols, such as Ethernet (or IEEE 802.3), ATP, Bluetooth, and TCP/IP, TDMA, CDMA, 3G, etc.; and/or (iv) encrypted to ensure privacy or prevent fraud in any of a variety of ways well known in the art.

It will be appreciated that as used herein, the terms system, subsystem, service, programmed logic circuitry, and the like may be implemented as any suitable combination of software, hardware, firmware, and/or the like. It also will be appreciated that the storage locations herein may be any suitable combination of disk drive devices, memory locations, solid state drives, CD-ROMs, DVDs, tape backups, storage area network (SAN) systems, and/or any other appropriate tangible computer readable storage medium. It also will be appreciated that the techniques described herein may be accomplished by having a processor execute instructions that may be tangibly stored on a computer readable storage medium.

As used herein, the term “non-transitory computer-readable storage medium” includes a register, a cache memory, a ROM, a semiconductor memory device (such as a D-RAM, S-RAM, or other RAM), a magnetic medium such as a flash memory, a hard disk, a magneto-optical medium, an optical medium such as a CD-ROM, a DVD, or Blu-Ray Disc, or other type of device for non-transitory electronic data storage. The term “non-transitory computer-readable storage medium” does not include a transitory, propagating electromagnetic signal.

When it is described in this document that an action “may,” “can,” or “could” be performed, that a feature or component “may,” “can,” or “could” be included in or is applicable to a given context, that a given item “may,” “can,” or “could” possess a given attribute, or whenever any similar phrase involving the term “may,” “can,” or “could” is used, it should be understood that the given action, feature, component, attribute, etc. is present in at least one embodiment, though is not necessarily present in all embodiments.

Embodiments of the present disclosure can be further defined by reference to the following non-limiting examples, which describe in detail systems and methods of the present disclosure. It will be apparent to those skilled in the art that many modifications, both to materials and methods, can be practiced without departing from the scope of the present disclosure.

All documents, patent, and patent applications cited herein are hereby incorporated by reference, and may be employed in the practice described herein.

### EXAMPLE USES OF THE VISUALIZING SYSTEM

### Example 1. Observing Changes in Taxonomic Structure

Samples were collected from two groups of swine (one control group and one treatment group provided with a high protein diet) by extracting DNA from the cecum after 30 days post-natal. These samples were sequenced using a shotgun metagenomic approach to produce OTUs. Changes in microbial community are often visualized using non-metric multi-dimensional scaling (e.g., Bray-Curtis distance) plots, but often clear differences in a treatment are not apparent (FIG. 4). However, when evaluating the network structure of the microbial community using tools and/or methods described herein, more apparent changes were found.

To construct the base network, each OTU served as an independent node and no edges connecting the nodes existed (FIG. 6A). To construct the edges that convert the network from static to dynamic, the inventors used Spearman Correlation data of each OTU to every other OTU. Briefly, OTU data from each group was separated and run through Spearman Correlations of each OTU to each other OTU. These Spearman Correlations served as the dynamic edges in the network. Under the control conditions, the Spearman Correlations reflected the strength and association of each microbe to every other microbe for the control group of swine. Under treated conditions, the Spearman Correlations reflected the strength and association of each microbe to every other microbe for the treatment group of swine. Thus, the dynamic edges reflected how the strength and association of each microbe to every other microbe were modulated by the treatment.

Filtering was done to select for a narrow range of correlation strength (r=0.6). This filtering revealed a characteristic structure and shape of the microbiome under control conditions (FIG. 6B). Simply by allowing the dynamic network to change, the inventors observed the new microbiome structure in swine that were treated (FIG. 6C). These edges created ball-like structures within the network. The network had a clearly different structure after the treatment was applied. These changes in the network structure are expected to change the potential flow of information and allow for understanding of keystone microbes.

Using network statistics like eigenvector centrality (an organism highly connected to its own sub-community) and betweenness centrality (organisms highly connected to different sub-communities), the inventors identified keystone organisms. Under control conditions, OTU630 had the largest eigenvector centrality at 0.85 while OTU539 had the largest betweenness centrality at 0.597. Changing to the treatment it was OTU164 that had the maximum eigenvector centrality (1.0) and OTU147 had the maximum betweenness centrality (0.064). In the treatment condition, OTU630 had a much lower eigenvector centrality (0.004) and OTU539 had a much lower betweenness centrality (0.002).

Distribution of connectivity of organisms within the network was analyzed by determining shift (ZiPi) distance. ZiPi distance is described in Olesen, Jens M et al. “The modularity of pollination networks.” Proceedings of the National Academy of Sciences of the United States of America vol. 104, 50 (2007): 19891-6. doi:10.1073/pnas.0706375104. Increasing P1 shows an increase connectivity to multiple groups in the network and an increasing Z1 shows an increasing connectivity to an individual group. FIG. 6D shows the distribution of connectivity of organisms within the network that experienced the largest shifts (ZiPi) distances.

Network structure was evaluated for “small-worldedness” by the factor S. S is described in “Network ‘Small-World-Ness’: A Quantitative Method for Determining Canonical Network Equivalence”, Humphries M D, Gurney K (2008) Network ‘Small-World-Ness’: A Quantitative Method for Determining Canonical Network Equivalence. PLOS ONE 3(4): e0002051. https://doi.org/10.1371/journal.pone.0002051. An S>>1 indicates a small-world effect. The strength of the small-world effect increased with treatment (FIG. 6E). This indicates that more organisms/nodes were tightly connected after the treatment than before.

### Example 2. Changes in Function

Samples were collected from two groups of swine (one control group and one treatment group provided with a high protein diet) by extracting DNA from the cecum after 21 days post-weaning. These samples were sequenced using a shotgun metagenomic approach. Reads were aligned to a vendor-provided database (public NCBI databases, KEGG, BRENDA, etc. may be used, alternatively) of annotated gene sequences to produce a count table of genes for each sample from both groups. Typical metabolism analysis collapses genes into unique pathways and looks at overall changes as shown in the heatmap (FIG. 7). However, this analysis is merely a high level overview of what is going on at a functional level. Available metabolic networks, for example, KEGG (www.genome.jp/kegg) and GOMixer (www.raeslab.org/omixer/visualisation/map), merely form the scaffold but don't facilitate any in-depth analysis. These available metabolic networks are also static and don't allow for transitions between different environments, times, or treatments that may be part of an experiment.

In contrast, systems and methods described herein allow deep functional analysis of metagenomics data sets. The inventors constructed a gene-level metabolic network where the nodes were metabolites and the edges were enzymatic reactions between the metabolites. Next, the gene counts were matched against the edges of the network and the relative abundance of gene counts of each edge were normalized to the control such that our control edges all had a weight equal to one (FIG. 9A). The static network graph was converted to a dynamic one and data from the additional treatments were added into the network. Looking at the treatment group, there was a clear increase in certain enzyme reactions and decrease in others, as shown in FIG. 9B (relative thickness of lines reflect relative abundance of gene/enzyme edges). There was a decrease in edges 197, 166, 167, and 168 between nodes/metabolites 0, 48, 75, 76, and 90 (bordered area top left; FIG. 9B). There was an increase in edges 232, 233, 235, 268, and 269 associated with nodes/metabolites 0, 28, 48, 106, 107, and 133 (bordered area in the center; FIG. 9B). Running statistics, results were embedded in the same graph and p-values of the treatment relative to the control were visualized. FIG. 9C shows the statistical significance of the change in the reaction relative to the control as a function of color (black->white or darker.->lighter). This shows that the above discussed decreases and increases in edges were indeed significant. Relating information about the treatment and the animals within the study, the inventors were able to associate these pathways as protagonistic or antagonistic to swine.

### Example 3. Integrated Taxonomic and Metabolic Analysis

In another trial, two groups of pigs were subjected to two different doses of an enzyme (muramidase). Samples were collected from the two groups of swine by extracting DNA from the cecum. These samples were sequenced using a shotgun metagenome approach and the results were interpreted using MetaPhlan2 for taxonomy and KEGG for function. The analysis further included a third control dataset obtained from a group of pigs that were not treated with the enzyme.

The enzyme treatment had an effect on the structure of the community by impacting two selected microorganisms differently (FIG. 11A: 0—Control, Low Dose, High Dose). Organism 1 increased while organism 2 decreased. The initial functional network visualized under control conditions showed a rather sparse network (FIG. 11B). Focusing on the effect of the added enzyme, changes in the pathway were visualized (FIG. 11C). Using the reactions between the nodes included in the bordered area in FIG. 11B, one enzymatic reaction is characterized as increasing (edge 200) and one enzymatic reaction is characterized as decreasing (edge 178). FIG. 11C. Going back to the database from which the identified microbial genes were identified, organism information was coupled to the functional annotations to understand how the shift in metabolism was being derived from these organisms. The two organisms' metabolic network were first visualized (FIG. 11D) with the nodes highlighted in FIGS. 11B and C placed near each other in the center. It is interesting to note that one of the two enzymatic reactions being focused on did not exist in one of the two organisms suggesting that the entire contribution of this reaction came from an individual organism. Visualizing the change in the enzymatic reactions of the same edges but from two different organisms explains the changes in FIG. 11C more thoroughly. The decrease in edge 178 was derived from a decrease in organism 2. While edge 200 does increase overall, organism 1 contributed to an increase while organism 2 decreased at that edge. The edge that was decreasing likely did not confer a benefit to the organism and therefore did not provide organism 2 any fitness advantage. Adding in the metabolite at node could increase the fitness benefit of edge 178 and increase the relative abundance of this organism. This example shows how dynamic visualization of metabolic networks integrated with taxonomic information allows for the development of molecules to selectively manipulate different organisms within the overall population.

### Example 4. Identification of Genes and/or Metabolites for Diagnostics Relative to a Control Condition

Post-weaning diarrhea is an affliction that affects piglets and can result in increases in mortality of the piglets. Identifying changes in microbiome and potential metabolites associated with post-weaning diarrhea can create an early warning diagnostic that could facilitate intervention and thus reduce mortality. In a study of post-weaning diarrhea, a cohort of piglets was tracked over 20 days. Samples were taken for microbiome sequencing, metabolite extraction, and a fecal score was recorded. A fecal score could be an integer between 0 (no diarrhea) to 3 (severe diarrhea). Fecal score is usually recorded currently by human observation.

After sequencing the microbiome and identifying genes present in the pathways, changes were be normalized to a fecal score of 0 (no diarrhea) to look for changes (FIG. 13A). Examining the network at a fecal score of 3, a large shift in pathways were seen to be both increasing (FIG. 13B; bottom right) and decreasing (FIG. 13B; top left). The increasing enzymatic reactions, edges 217 and 220 were identified as candidate biomarkers and can be further examined as a biomarker associated with increasing fecal score.

The systems and methods described herein can also be used to examine decreased pathways to identify bottlenecks that can become potential treatment goals for substitution therapy with probiotic containing missing/decreased pathways, or to identify upstream metabolic markers that can serve as diagnostic markers. To prove that the systems and methods described herein can be used to identify metabolic markers and how the metabolic network is directly tied to these metabolites, metabolite levels were integrated into the metabolic network. Focusing on an area where reactions were decreasing (FIG. 13C), the inventors expected that an accumulation of a metabolite would occur. With measured metabolites shown in red (FIGS. 13D and E), one metabolite could be visually identified as decreasing that is also adjacent to decreased reactions (FIG. 13E). Upstream of this metabolite where enzymatic reactions were still present, there was an accumulation of a metabolite. This sharp change in abundance of enzymatic reactions is known as a bottleneck (FIGS. 13B, 13D, and 13E), where without measurement of metabolite data (FIG. 13B), one can generate hypothetical metabolic markers as targets. However, the step of incorporating measurements of metabolites (FIG. 13E) solidified the understanding of where the accumulation was actually occurring.

### Example 5. Developing Internal Standards to Monitor Fluctuations in Microbiome

Samples were collected from four groups of swine (one control group and three treatments groups (arachidonic acid, docosahexenoic acid, and a combination) by extracting DNA from the cecum after 30 days post-natal. The samples were sequenced to produce OTU's. The samples were sequenced using a shotgun metagenomic approach. Sequence reads were aligned to a database of annotated gene sequences to produce a count table of genes for each sample from all groups. The inventors then constructed a gene-level metabolic network where the nodes are metabolites and the edges are enzymatic reactions between the metabolites. Next, the gene counts were matched against the edges of the network and the relative abundance of gene counts of each edge were normalized to control such that the control edges all had a weight equal to one (FIG. 15A). The static network graph was converted to a dynamic one and data from the additional treatments was added into the network. In this experiment, a desired phenotype was observed that was correlated with a change in a specific gene (FIG. 15B). Compared to control conditions, there was an edge increase due to a specific treatment that had a positive effect on the host animal. This gene/enzyme/reaction was therefore selected as a candidate diagnostic marker. Observing this change relative to the untreated control, there was a need to identify an internal standard that can be used in situations where every animal is being treated to monitor the effectiveness of the treatment without having to run a control untreated group of animals. A good control gene/reaction/enzyme that can serve as an internal standard is a gene/reaction/enzyme whose abundance is relatively constant and does not fluctuate. Additional criteria to consider for selecting an internal control for a particular candidate marker can includes competing pathway(s), reaction(s). To identify such an internal control, all edges/genes/reactions were examined for their standard deviation (FIG. 15C). Using an edge with the lowest standard deviation as the internal control, the changes in the candidate diagnostic marker level were more efficiently visualized and monitored internal to a single treatment (FIG. 15D).

### Example 6. Using Host Response Identified Targets to Infer Microbiota Produced Signaling Metabolites or Pathway Shared Intermediates

Molecules produced by the microbiome upon application of exogenous products majorly impact gene expression in host intestinal cells. It is known by people skilled in the art how to identify gene expression modification within eukaryotic cells. The systems and methods disclosed herein can be deployed to use the eukaryotic fluctuating internal host targets to identify corresponding dynamically changing microbiota metabolites. These microbiota metabolites identified by the dynamic microbiota model can either compensate for fluctuating intermediates within the host cell after uptake (direct effect on host target) (targeted application of the dynamic microbiota model), or induce indirectly a metabolic answer/response from the host (ligand to receptor classical cascade signaling) (untargeted approach of the dynamic microbiota model).

Proline metabolism provides an example of the theoretical concept of metabolic compensation between host and microbiome (direct effect on host target). FIG. 16, shows the combined proline metabolic network of host and its microbiome. Red and blue boxes indicate host and microbiota genes/enzymes, respectively. The microbiome is capable of converting proline into ornithine, whereas the host cells do not express one of the three enzymes that mediate the transformation of proline into ornithine. Ornithine produced by the microbiome, however, can be utilized by the host cells.

The inventors collected RNA samples from a set of pigs that were on a diet to induce diarrhea as a simulation for post-weaning diarrhea. Host metabolism was combined with the microbiota to derive connection between potential signaling molecules (above).

Identification of microbiota compounds correlated with the identified host targets. Using a dynamic model described herein, the user can identify shared metabolites fluctuating accordingly to the host targets using KEGG pathways. Host responses and direct host connections to metabolites were embedded in the dynamic metabolite network to visualize the connections and the strength of the connections between the microbiome and the host. FIG. 17A. The host response to putrescine is represented by the largest node 1702 ornithine and the molecule putrescine is represented by the connected large node 1704. The dynamic network further provided a multi-dimensional view into the microbiome-host connection. In FIG. 17B, the width of the lines between nodes is the gene abundance relative to the control, and the color gradient represents the q-value (lower is more significant). This shows in the treated case that the microbiome has significant connections to putrescine and this is reflected in the connection to the host.

Using a dynamic model described herein, the user can further identify fluctuating compounds involved into signaling based on a ligand-receptor concept, for example, acting on inflammation and immunity pathway.

Although particular embodiments have been described above, a person of skill in the art having been provided with this disclosure, would appreciate aspects of the different embodiments may be used in various combinations to realize still other embodiments of a technique and/or kiosk for recycling electronic devices.

While the embodiments presented herein have been described in detail, the foregoing description is in all aspects illustrative and not restrictive. It is understood that numerous other modifications and variations can be devised without departing from the scope of the disclosed embodiments.

1-37. (canceled)

38. A system for visualizing microbiome data, comprising:
a memory storing a database of said microbiome data representing microbiomes, in a plurality of samples acquired at respective times, from each of a plurality of groups of animals including at least one control group and at least one treatment group;
a display;
a processor configured to:
identify respective microbes and/or genes in the microbiome data stored in the database;
generate a network comprising nodes interconnected by edges in the memory, each node representing one or more identified microbes or one or more microbial metabolites, and each edge of the network representing an association between a respective pair of the one or more identified microbes or a reaction mediated between two metabolites by an enzyme encoded in the one or more identified genes, wherein at least some nodes and edges of the network are each associated with a condition attribute identifying one of said plurality of groups from and/or a timestamp attribute identifying a time of one of said samples; and
responsive to interactive input, dynamically update the network displayed on the display in accordance with a filtering, of the microbiome data, based at least on the condition attribute and/or the timestamp attribute associated with respective nodes and/or edges in the network.

39. The system according to claim 38, wherein the network includes all microbes identified for the microbiome in the microbiome data, each microbe in the microbiome data being represented by a node of the network.

40. The system according to claim 38, wherein each node of the network represents one of an operational taxonomic unit (OTU), a microbe ID, a taxonomy, or a metabolite.

41. The system according to claim 38, wherein a first node and a second node in the network each represents a respective microbe and an edge between the first and second nodes represents a statistical correlation, observation, or a characteristic associating organisms together.

42. The system according to claim 41, wherein the processor is further configured to:
calculate a correlation, in the microbiome data, of the microbes represented by the first and second nodes; and
indicate at least some of the calculated correlation in the displayed network.

43. The system according to claim 38, wherein a first node and a second node in the network each represents a respective metabolite and an edge between the first and second nodes represents a gene annotation, sequence, or a reaction between two metabolites.

44. The system according to claim 38, wherein nodes of the network are constrained to exist within separate metabolic clusters representing respective organisms such that respective multiple metabolic networks each represent a different microbe and connections between metabolic clusters are connected through metabolite nodes deemed as extracellular.

45. The system according to claim 38, wherein the microbiome data includes taxonomic data derived from 16S marker gene surveys, metagenomic sequencing, or another technique allowing identification, delineation, and counting of separate organisms.

46. The system according to claim 38, wherein the microbiome data includes metabolic pathway data derived from predicted metagenomes, shotgun metagenomic sequencing, or another technique that allows identification, delineation, and counting of separate genes.

47. The system according to claim 38, wherein nodes in the network each represents a respective microbe, and wherein the processor is further configured to, in response to receiving interactive input, performing taxonomic restructuring of the network by applied condition.

48. The system according to claim 47, wherein the processor is further configured to, in response to receiving interactive input, perform said taxonomic restructuring providing identification of conditions to selectively increase or decrease relative abundance of selected organisms in the microbiome.

49. The system according to claim 48, wherein the processor is further configured to, in response to receiving interactive input, perform said taxonomic restructuring providing identification of conditions that maximize commensal-istic conditions that benefit a host of the sample or minimize competition that causes conditions detrimental to the host.

50. The system according to claim 38, wherein nodes in the network each represents a respective microbe, and wherein the processor is further configured to, in response to receiving interactive input, perform network restructuring of the network.

51. A method for visualizing microbiome data, comprising:
identifying respective microbes and/or genes in microbiome data stored in a database, wherein the database of said microbiome data represents microbiomes, in a plurality of samples acquired at respective times, from each of a plurality of groups of animals including at least one control group and at least one treatment group;
generate a network comprising nodes interconnected by edges in the memory, each node representing one or more identified microbes or one or more microbial metabolites, and each edge of the network representing an association between a respective pair of the one or more identified microbes or a reaction mediated between two metabolites by an enzyme encoded in the one or more identified genes, wherein at least some nodes and edges of the network are each associated with a condition attribute identifying one of said plurality of groups from and/or a timestamp attribute identifying a time of one of said samples; and
responsive to interactive input, dynamically update the network displayed on a display in accordance with a filtering, of the microbiome data, based at least on the condition attribute and/or the timestamp attribute associated with respective nodes and/or edges in the network.

52. A non-transitory computer readable storage medium storing instructions, which, when executed by one or more processors of a computer, causes the computer to perform operations including:
identifying respective microbes and/or genes in microbiome data stored in a database, wherein the database of said microbiome data represents microbiomes, in a plurality of samples acquired at respective times, from each of a plurality of groups of animals including at least one control group and at least one treatment group;
generating a network comprising nodes interconnected by edges in the memory, each node representing one or more identified microbes or one or more microbial metabolites, and each edge of the network representing an association between a respective pair of the one or more identified microbes or a reaction mediated between two metabolites by an enzyme encoded in the one or more identified genes, wherein at least some nodes and edges of the network are each associated with a condition attribute identifying one of said plurality of groups from and/or a timestamp attribute identifying a time of one of said samples; and
responsive to interactive input, dynamically updating the network displayed on a display in accordance with a filtering, of the microbiome data, based at least on the condition attribute and/or the timestamp attribute associated with respective nodes and/or edges in the network.
